(19) United States
(12) Patent Application Publication
(10) Pub. No.: US 2025/0026802 A1
(43) Pub. Date: Jan. 23, 2025

(54) CHIMERIC ADIPONECTIN POLYPEPTIDES, EXTRACELLULAR VESICLE COMPRISING THE SAME, AND USES THEREOF

(71) Applicants: CILOA, Montpellier (FR); JERID, Saint André de Sangonis (FR)

(72) Inventors: Zaïne El Abiddine Robert MAMOUN, Saint-André-de-Sangonis (FR); Bernadette Nadine TRENTIN, Saint-André-de-Sangonis (FR); Katarzyna POLAK, Montpellier (FR)

(73) Assignees: CILOA, Montpellier (FR); JERID, Saint André de Sangonis (FR)

(21) Appl. No.: 18/714,336

(22) PCT Filed: Dec. 6, 2022
(86) PCT No.: PCT/EP2022/084653

§ 371 Date: May 29, 2024

(30) Foreign Application Priority Data
Dec. 6, 2021 (EP) 21306717.6

(51) Int. Cl. C07K 14/575; A61K 38/00; C07K 14/705; C07K 14/715

(52) U.S. Cl. CPC C07K 14/575; C07K 14/70517; C07K 14/7151; A61K 38/00; C07K 2319/03; C07K 2319/033

(57) ABSTRACT

Chimeric polypeptides having an amino acid sequence of adiponectin and an amino acid sequence of a transmembrane domain of a transmembrane protein, nucleic acids encoding these chimeric polypeptides, and extracellular vesicles including these chimeric polypeptides. Also, the use of these extracellular vesicles as a medicament, and in particular, for treating various diseases, disorders or conditions.

### FIELD OF INVENTION

The present invention relates to chimeric polypeptides comprising adiponectin, nucleic acids encoding said chimeric polypeptides, and extracellular vesicles comprising said chimeric polypeptides. It further relates to the use of said extracellular vesicles as a medicament, and in particular, for treating various diseases.

### BACKGROUND OF INVENTION

Adipose tissue is an endocrine organ that secretes a wide variety of bioactive proteins (adipokines) and lipids, among which adiponectin is one of the most abundant adipokines. Adiponectin is a 244 amino acid cytokine, that is mainly found in oligomeric complexes. Adiponectin is known to exert beneficial effects in various human and animal conditions, including insulin resistance, cardiovascular disease, inflammatory conditions and cancer. Thus, adiponectin is a promising candidate for drug development of various diseases.

Despite this promising aspect, there are currently no adiponectin therapies available for clinical testing. Indeed, large-scale production of adiponectin native functional forms, including post-translational modifications and proper highly multimerized complex assembly, is a daunting task. Combined with the short half-life of adiponectin, no production strategy supplying high levels of bioactive adiponectin at a reasonable price exists. Therefore, alternative approaches providing multimeric native active forms of adiponectin are then awaited.

In the present invention, the inventors have found that adiponectin can be produced in high amounts and in native highly multimerized forms when anchored in extracellular vesicles, and that these adiponectin-rich extracellular vesicles can be purified and stored in characterized and qualified batches. Such standardized material open the way for promising strategies to treat the various human conditions that benefit from adiponectin, such as, for example, diabetes, obesity and associated metabolic disease, insulin resistance, cardiovascular disease, inflammatory conditions and cancer.

### SUMMARY

The present invention relates to a chimeric polypeptide comprising, in any order:
i) an amino acid sequence of adiponectin, preferably of wild type adiponectin,
ii) an amino acid sequence of a transmembrane domain of a transmembrane protein, and
iii) optionally, an amino acid sequence of a peptide interacting with the Endosomal Sorting Complexes Required for Transport (ESCRT) cellular machinery.

In one embodiment, the chimeric polypeptide further comprises at least one linker between the amino acid sequence of adiponectin and the amino acid sequence of the transmembrane domain.

In one embodiment, the chimeric polypeptide comprises, from N- to C-terminal, the components iii), ii) and i).

In one embodiment, the chimeric polypeptide further comprises a sub-membrane targeting domain, preferably wherein sub membrane targeting domain is linked to an anchoring molecule, preferably wherein the anchoring molecule is a fatty acid.

In one embodiment, the transmembrane domain is selected from the group comprising the transmembrane domain of CD40L and a transmembrane domain of CD8; preferably with SEQ ID NO: 34 and SEQ ID NO: 36, respectively more preferably with SEQ ID NO: 35 and SEQ ID NO: 37, respectively.

In one embodiment, the pilot peptide comprises at least one YxxL motif with SEQ ID NO: 1 or DYxxL motif with SEQ ID NO: 4, and at least one PxxP motif with SEQ ID NO: 8, in which “x” represents any amino acid residue.

In one embodiment, the pilot peptide comprises an amino acid sequence with SEQ ID NO: 30 or a variant thereof, wherein the variant of SEQ ID NO: 30 retains at least three YxxL and/or DYxxL motifs with SEQ ID NO: 1 and SEQ ID NO: 4, respectively; and at least four PxxP motifs with SEQ ID NO: 8; wherein “x” represents any amino acid residue.

The present invention also relates to a nucleic acid encoding the chimeric polypeptide of the invention.

The present invention also relates to an extracellular vesicle comprising the chimeric polypeptide of the invention, preferably wherein:
the transmembrane domain of the chimeric polypeptide is anchored in the extracellular vesicle lipid bilayer; and
the adiponectin of the chimeric polypeptide is exposed at the outer surface of the extracellular vesicle.

In one embodiment, the extracellular vesicle is an exosome, preferably having a diameter ranging from about 30 nm to about 120 nm.

The present invention also relates to a population of extracellular vesicles of the invention, optionally further comprising soluble adiponectin.

In one embodiment, the extracellular vesicle of the invention or the population of extracellular vesicles of the invention, are purified, preferably are ultra-purified.

The present invention also relates to the extracellular vesicle of the invention or to the population of extracellular vesicles of the invention, for use as a medicament.

The present invention also relates to the extracellular vesicle of the invention or to the population of extracellular vesicles of the invention, for use in treating a disease, disorder or condition selected from the group comprising diabetes, obesity, insulin resistance, diseases related to insulin resistance or deficiency, hypertension, dyslipidemia, hyperuricemia, atherosclerosis (including coronary artery disease, stroke and peripheral artery disease), fibrosis, inflammatory pulmonary diseases, nephrotic disease, sleep apnea, dry eye diseases, inflammatory ocular diseases, gastritis and gastro esophageal reflux disease, inflammatory bowel disease, pancreatitis, osteoporosis, and inflammatory bone and joint diseases.

The present invention also relates to an extracellular vesicle harboring adiponectin exposed at its outer surface, or a population thereof, for use in treating a disease, disorder or condition selected from the group comprising diabetes, obesity, insulin resistance, diseases related to insulin resistance or deficiency, hypertension, dyslipidemia, hyperuricemia, atherosclerosis (including coronary artery disease, stroke and peripheral artery disease), fibrosis, nephrotic disease, sleep apnea, dry eye diseases, inflammatory ocular diseases, gastritis and gastro-esophageal reflux disease, inflammatory bowel disease, pancreatitis, osteoporosis, and inflammatory bone and joint diseases.

In one embodiment, adiponectin is a recombinant adiponectin, optionally fused to lactadherin or a functional C1 and/or C2 domain thereof.

The present invention also relates to a method of diagnosing obesity, insulin resistance, or a disease related to insulin resistance or deficiency in a subject, comprising the steps of:
a) measuring the level or amount of adiponectin-associated small extracellular vesicles in a sample previously retrieved from the subject;
b) comparing the level or amount determined at step a) with a reference level or amount of adiponectin-associated small extracellular vesicles, wherein the reference level or amount was pre-determined in a sample from a reference subject known not to be suffering from obesity or insulin resistance, or in a pool of samples from more than one reference subjects, all known not to be suffering from obesity or insulin resistance;
c) concluding that the subject is affected with obesity, insulin resistance, or a disease related to insulin resistance or deficiency, when the level or amount determined at step a) is lower than the reference level or amount.

### Definitions

In the present invention, the following terms have the following meanings:

“About” preceding a figure means plus or less 10% of the value of said figure.

“Adiponectin” (also known as “30 kDa adipocyte complement-related protein”, “Adipocyte complement-related 30 kDa protein”, or in short, “ACRP30”) is a protein that is primarily derived from adipocytes. As used herein, the term “adiponectin” includes adiponectin from any species that produces adiponectin. In humans, adiponectin is encoded by the ADIPOQ gene (also referred to as ACDC, ACRP30, APMI, or GBP28 gene). This gene encodes an adiponectin precursor with SEQ ID NO: 33, which is processed in vivo into its mature form with SEQ ID NO: 31.

“CD8” refers to a transmembrane glycoprotein that serves as a co-receptor for the T cell receptor (TCR). In humans, CD8 transmembrane domain comprise an amino acid sequence with SEQ ID NO: 36.

“CD40 ligand” (also called “CD40L” or “CD154”) refers to a transmembrane protein, member of the tumor necrosis factor (TNF) superfamily. In humans, CD40L transmembrane domain comprise an amino acid sequence with SEQ ID NO: 34.

“Chimeric”, when referring to a polypeptide, refers to a polypeptide that combines several domains of at least two different types by their function and/or by their cellular localization, wherein the at least two of these domains come either from distinct proteins of the same or different species, or from the same protein of different species.

“Diabetes” refers to a metabolic disease characterized by a chronic excess of sugar (glucose) in the blood, also referred to as “diabetes mellitus”. One of the criteria used to diagnose diabetes is a fasting glycemia level greater than 1.26 g/L of blood (or about 7 mmol/L of blood). There are several types of diabetes:
“type 1 diabetes”, also called “juvenile diabetes”, is characterized by the disappearance in children or young adults of the B cells of the islets of Langerhans of the pancreas, which produce insulin (autoimmune disease); and
“type 2 diabetes”, also called “noninsulin-dependent diabetes”, is characterized by the gradual onset of insulin resistance (i.e., when the response of insulin-sensitive cells to insulin is diminished). Insulin resistance is symptomized by the decrease of glucose uptake by fat tissues and muscles and the decrease of hepatic glucose production inhibition. At a more advanced stage, type 2 diabetes can lead to insulin deficiency, i.e., a failure to synthesize insulin by endocrine pancreatic cells.

“Disease related to insulin resistance or deficiency” or “disease related to glucose intolerance” refers to several metabolic diseases including type 2 diabetes, metabolic syndrome, but also cardiovascular disease (Ford, 2005. Diabetes Care. 28 (7): 1769-78), non-alcoholic fatty liver disease (Bugianesi et al., 2010. Curr Pharm Des. 16 (17): 1941-51), polycystic ovary syndrome (PCOS) (Diamanti-Kandarakis, 2006. Endocrine. 30 (1): 13-7), Alzheimer's disease (Watson & Craft, 2003. CNS Drugs. 17 (1): 27-45) and cancer (Arcidiacono et al., 2012. Exp Diabetes Res. 2012:789174).

“Domain”, when referring to a protein or a polypeptide, refers to a region having a structural and/or functional property for said protein or polypeptide. As used herein, a “transmembrane domain” refers to a functional region of a protein or polypeptide that spans the phospholipid bilayer of a biological membrane.

“ESCRT” or “endosomal sorting complexes required for transport” refers originally to a cellular machinery made up of five multi-subunit protein complexes, which act cooperatively at specialized endosomes to facilitate the movement of specific cargoes from the limiting membrane into vesicles that bud into the endosome lumen. This machinery is hijacked by several envelope viruses to bud from cellular membranes, including the plasma membrane.

“Exosome” refers to an extracellular vesicle that is produced in the endosomal compartment of eukaryotic cells (Théry et al., 2018. J Extracell Vesicles. 7 (1): 1535750; Yáñez-Mó et al., 2015. J Extracell Vesicles. 4:27066; van Niel et al., 2018. Nat Rev Mol Cell Biol. 19 (4): 213-228). Typically, exosomes harbor at their surface the CD81, CD63 and CD9 markers.

“Expression vector” refers to a vector capable of directing expression of a nucleic acid sequence of interest (such as, e.g., a nucleic acid according to the present invention) in an appropriate host cell, comprising a promoter operatively linked to the nucleic acid sequence of interest, itself operatively linked to a termination sequence.

“Extracellular vesicle” refers to any vesicle composed of a lipid bilayer that is naturally released from a cell and comprises a cytosolic fraction of said cell. This expression in particular includes vesicles secreted into the extracellular space, i.e., “exosomes”.

“Insulin resistance” refers to the inability of a known quantity of exogenous or endogenous insulin to increase glucose uptake and utilization in a subject as much as it does in a normal population.

“Isolated” and any declensions thereof, as well as “purified” and any declensions thereof, are used interchangeably, and mean that a molecular entity to which it refers (e.g., a polypeptide, a nucleic acid, an extracellular vesicle, etc.) is substantially free of other components (i.e., of contaminants) found in the natural environment in which said molecular entity is normally found. Preferably, an isolated or purified molecular entity (e.g., an isolated or purified polypeptide, an isolated or purified nucleic acid, an isolated or purified extracellular vesicle, etc.) is substantially free of other molecular entities with which it is associated in a cell. By “substantially free”, it is meant that said isolated or purified molecular entity represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Obesity” (or overweight) refers to a chronic disease (as recognized by the World Health Organization since 1997), defined as an abnormal or excessive accumulation of body fat which can be harmful to health. Still according to the World Health Organization, the definition of obesity is based on the measurement of the body mass index

$$( B M I = \frac{m a s s}{{height}^{2}} ) ,$$

classifying the disease into 3 levels: “overweight” if 25 kg/m²<BMI<30 kg/m²; “obesity” if 30 kg/m²<BMI<40 kg/m²; and “morbid obesity” if 40 kg/m²<BMI.

“Pilot peptide” refers to a peptide that interact with ESCRT proteins. Said pilot peptide is capable of being addressed to membrane vesicles, in particular to exosome-forming vesicles, or to the cell compartment(s) involved in the formation of membrane vesicles, and in particular of exosome-forming vesicles in eukaryotic cells.

“Subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease.

“Sub-membrane targeting domain” or “membrane targeting domain” or “membrane recruitment domain” are used interchangeably to refer to a domain capable of, in a cell and in particular in a eukaryotic cell (e.g., in an exosome-producing cell), to anchor itself to a cell membrane and/or a vesicular membrane without being inserted into said membrane, said anchoring being achieved by means of one or more anchoring molecule(s) and/or by interactions (e.g., electrostatic interactions) between the sub-membrane targeting domain and the membrane. In a particular embodiment, the sub-membrane targeting domain is capable of binding to, or interacting with, the inner surface of the cell membrane (i.e., the cytoplasmic side of the cell membrane) and/or with the inner surface of vesicular membranes (i.e., the lumen side of the vesicular membrane).

“Therapeutically effective amount” refers to the level or amount of a chimeric polypeptide, nucleic acid, extracellular vesicle, population of extracellular vesicles, composition, pharmaceutical composition, medicament, etc. that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Transmembrane protein” refers to a protein which comprises at least one transmembrane domain, allowing it to be anchored in the phospholipid bilayer of a biological membrane. A transmembrane domain is generally hydrophobic-helical, and can contain several, in particular 2, 3, 4, 5, 6, 7, 8, 9 or 10, or even 20 or more, hydrophobic α-helices. It can also be arranged in a β-sheet, e.g., in a β-barrel structure typically composed of 8 to 22 β-strands. The transmembrane proteins can also be classified according to the position of the N- and C-terminal on the different sides of the lipid layers: Types I, II, III and IV. Type I transmembrane proteins are anchored to the lipid membrane with a stop-transfer anchor sequence and have their N-terminal domains targeted to the endoplasmic reticulum (ER) lumen during synthesis (and the extracellular space, if mature forms are located on cell membranes). Type II and III are anchored with a signal-anchor sequence, with type II being targeted to the ER lumen with its C-terminal domain, while type III have their N-terminal domains targeted to the ER lumen. Type IV is subdivided into IV-A, with their N-terminal domains targeted to the cytosol and IV-B, with an N-terminal domain targeted to the lumen.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. In one embodiment, a subject is successfully “treated” for a disease, disorder, or condition if, after receiving a therapeutic amount of a chimeric polypeptide, nucleic acid, extracellular vesicle, population of extracellular vesicles, composition, pharmaceutical composition, medicament, etc., the subject shows at least one of the following: relief to some extent of one or more of the symptoms associated with the disease, disorder, or condition to be treated; reduced morbidity and mortality; and improvement in quality of life issues. The above parameters for assessing successful treatment and improvement in the disease are readily measurable by routine procedures familiar to a physician.

“Vector” refers to a nucleic acid capable of transporting a nucleic acid of interest (such as, e.g., a nucleic acid according to the present invention) to which it has been linked. Vectors capable of directing the expression of a nucleic acid of interest (such as, e.g., a nucleic acid according to the present invention) are referred to as “expression vectors”. In general, expression vectors are in the form of plasmids. Herein, the terms “plasmid” and “vector” are used interchangeably. However, other forms of expression vectors, which serve equivalent functions, are also encompassed under the term vector.

### DETAILED DESCRIPTION

The present invention relates to a chimeric polypeptide comprising, in any order:
i) an amino acid sequence of adiponectin,
ii) an amino acid sequence of a transmembrane domain of a transmembrane protein, and
iii) optionally, an amino acid sequence of a peptide interacting with the Endosomal Sorting Complexes Required for Transport (ESCRT) cellular machinery.

In one embodiment, components i), ii) and iii) are organized in the chimeric polypeptide from N- to C-terminal or from C- to N-terminal.

According to the invention, the chimeric polypeptide comprises an amino acid sequence of adiponectin.

In one embodiment, the amino acid sequence of adiponectin comprises or consists of the amino acid sequence of a wild-type adiponectin.

In one embodiment, the amino acid sequence of adiponectin comprises or consists of the amino acid sequence of a mutant adiponectin.

In one embodiment, the amino acid sequence of adiponectin comprises or consists of the amino acid sequence of the wild-type human adiponectin. In one embodiment, the wild-type human adiponectin comprises or consists of the amino acid sequence with SEQ ID NO: 33, or a variant thereof.

SEQ ID NO: 33
MLLLGAVLLLLALPGHDQETTTQGPGVLLPLPKGACTGWMAGIPGHPGHN
GAPGRDGRDGTPGEKGEKGDPGLIGPKGDIGETGVPGAEGPRGFPGIQGR
KGEPGEGAYVYRSAFSVGLETYVTIPNMPIRFTKIFYNQQNHYDGSTGKF
HCNIPGLYYFAYHITVYMKDVKVSLFKKDKAMLFTYDQYQENNVDQASGS
VLLHLEVGDQVWLQVYGEGERNGLYADNDNDSTFTGFLLYHDTN

Said wild-type human adiponectin comprises a peptide signal with amino acid sequence SEQ ID NO: 32, that is typically cleaved in vivo to form the mature form of the wild-type human adiponectin.

| SEQ ID NO: 32 | |
|---|---|
| | MLLLGAVLLLLALPGHDQ |

Hence, in one embodiment, the amino acid sequence of adiponectin comprises or consists of the amino acid sequence of the mature form of the wild-type human adiponectin with SEQ ID NO: 31, i.e, wherein the peptide signal with SEQ ID NO: 32 has been cleaved from the wild-type human adiponectin with SEQ ID NO: 33.

SEQ ID NO: 31
ETTTQGPGVLLPLPKGACTGWMAGIPGHPGHNGAPGRDGRDGTPGEKGEK
GDPGLIGPKGDIGETGVPGAEGPRGFPGIQGRKGEPGEGAYVYRSAFSVG
LETYVTIPNMPIRFTKIFYNQQNHYDGSTGKFHCNIPGLYYFAYHITVYM
KDVKVSLFKKDKAMLFTYDQYQENNVDQASGSVLLHLEVGDQVWLQVYGE
GERNGLYADNDNDSTFTGFLLYHDTN

In one embodiment, the amino acid sequence of adiponectin comprises or consists of the amino acid sequence with SEQ ID NO: 31, SEQ ID NO: 33, or a variant thereof.

In one embodiment, a variant of the amino acid sequence with SEQ ID NO: 31 or SEQ ID NO: 33 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 31 or SEQ ID NO: 33.

Additionally or alternatively, a variant of the amino acid sequence with SEQ ID NO: 31 or SEQ ID NO: 33 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 31 or SEQ ID NO: 33.

According to the invention, the chimeric polypeptide comprises an amino acid sequence of a transmembrane domain of a transmembrane protein.

In one embodiment, the transmembrane domain is that of a transmembrane protein from any organisms, including mammals, viruses and bacteria.

In one embodiment, the transmembrane domain is that of a transmembrane protein selected from the group comprising or consisting of human proteins, non-human animal proteins, pathogenic organism or agent proteins (in particular viral proteins, bacterial proteins, parasite proteins), or tumor cell proteins.

In one embodiment, the transmembrane domain is that of a transmembrane protein of type I. Said transmembrane domain of a transmembrane protein of type I may be used in a chimeric polypeptide as described hereinabove, wherein the components i), ii) and iii), when present, are organized in the chimeric polypeptide from N- to C-terminal (such as construct 1 of FIG. 1).

In one embodiment, the transmembrane domain is that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In one embodiment, the transmembrane domain is that of Influenza virus' hemagglutinin transmembrane protein.

In one embodiment, the transmembrane domain is that of CD8.

In one embodiment, the transmembrane domain is that of a transmembrane protein of type II. Said transmembrane domain of a transmembrane protein of type II may be used in a chimeric polypeptide as described hereinabove, wherein the components i), ii) and iii), when present, are organized in the chimeric polypeptide from C- to N-terminal (such as constructs 2, 3, 4, 5 of FIG. 1).

In one embodiment, the transmembrane domain is that of Influenza virus' neuraminidase transmembrane protein.

In one embodiment, the transmembrane domain is that of CD40 ligand (CD40L).

In one embodiment, the transmembrane domain is that of CD40 ligand (CD40L). or CD8.

In one embodiment, the transmembrane domain of CD40L comprises or consists of an amino acid sequence with SEQ ID NO: 34, or a variant thereof. In one embodiment, the transmembrane domain comprises or consists of an amino acid sequence with SEQ ID NO: 35, or a variant thereof.

SEQ ID NO: 34
IFMYLLTVFLITQMIGSALFAVYLH
SEQ ID NO: 35
MIETYNQTSPRSAATGLPISMKIFMYLLTVFLITQMIGSALFAVYLHRRL
DK

In one embodiment, the transmembrane domain of CD8 comprises or consists of an amino acid sequence with SEQ ID NO: 36, or a variant thereof. In one embodiment, the transmembrane domain comprises or consists of an amino acid sequence with SEQ ID NO: 37, or a variant thereof.

| SEQ ID NO: 36 | |
|---|---|
| | IYIWAPLAGICVALLLSLIITLICYH |
| SEQ ID NO: 37 | |
| | DIYIWAPLAGICVALLLSLIITLICYHR |

In one embodiment, a variant of the amino acid sequence with SEQ ID NO: 34, 35, 36 or 37 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 34, 35, 36 or 37.

Additionally or alternatively, a variant of the amino acid sequence with SEQ ID NO: 34, 35, 36 or 37 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 34, 35, 36 or 37.

In one embodiment, the chimeric polypeptide further comprises an amino acid sequence of a peptide interacting with the Endosomal Sorting Complexes Required for Transport (ESCRT) cellular machinery; otherwise known as “Pilot Peptide”.

In one embodiment, said pilot peptide is capable of being addressed to the membrane vesicles, in particular to the exosome-forming vesicles, or to the cell compartment(s) involved in the formation of the membrane vesicles, and in particular the exosome-forming vesicles in eukaryotic cells.

When integrated into a chimeric polypeptide, such as the chimeric polypeptide of the invention, the pilot peptide enables the addressing of said chimeric polypeptide to the membrane vesicles and/or to their location(s) of formation, and in particular enables the addressing of said chimeric polypeptide to the membrane of membrane vesicles, such that said polypeptide can be secreted by a cell in association with the membrane vesicles (in particular exosomes).

Pilot peptides which interact with ESCRT proteins have been described in granted patents EP 2 268 816, and U.S. Pat. No. 9,546,371, the relevant content of which is incorporated herein by reference.

In one embodiment, the pilot peptide comprises at least one YxxL motif (SEQ ID NO: 1), in which “x” represents any amino acid residue. In particular, it may comprise one, two or three YxxL motifs with SEQ ID NO: 1.

In one embodiment, said YxxL motif or one of the YxxL motifs of the pilot peptide may, for example, be YINL (SEQ ID NO: 2) or YSHL (SEQ ID NO: 3).

In one embodiment, the pilot peptide comprises a DYxxL motif (SEQ ID NO: 4), in which “x” represents any amino acid residue.

In one embodiment, said DYxxL motif may, for example, be DYINL (SEQ ID NO: 5).

Alternatively or additionally, the pilot peptide comprises at least one motif equivalent to a YxxL motif (SEQ ID NO: 1), for example, a YxxF motif (SEQ ID NO: 6), in which “x” represents any amino acid residue.

Alternatively or additionally, the pilot peptide comprises at least one motif equivalent to a DYxxL motif (SEQ ID NO: 4), for example, a DYxxF motif (SEQ ID NO: 7), in which “x” represents any amino acid residue.

In one embodiment, the pilot peptide further comprises at least one PxxP motif (SEQ ID NO: 8), in which “x” represents any amino acid residue. In particular, it may comprise one, two, three or four PxxP motifs with SEQ ID NO: 8.

In one embodiment, said PxxP motif or one of the PxxP motifs of the pilot peptide is PSAP (SEQ ID NO: 9) or PTAP (SEQ ID NO: 10).

In one embodiment, the pilot peptide comprises at least one YxxL motif with SEQ ID NO: 1 or DYxxL motif with SEQ ID NO: 4, and at least one PxxP motif with SEQ ID NO: 8.

In one embodiment, the pilot peptide consists of an amino acid sequence having one, two or three YxxL and/or DYxxL motif(s) with SEQ ID NO: 1 or SEQ ID NO: 4, respectively; and one, two, three or four PxxP motif(s) with SEQ ID NO: 8.

In one embodiment, the pilot peptide consists of an amino acid sequence having three YxxL and/or DYxxL motifs with SEQ ID NO: 1 or SEQ ID NO: 4, respectively; and four PxxP motifs with SEQ ID NO: 8.

In one embodiment, the YxxL motif with SEQ ID NO: 1 or at least one of the YxxL motifs with SEQ ID NO: 1, when more than one, is located downstream, i.e., in a C-terminal position, with respect to the one or more PxxP motif(s) with SEQ ID NO: 8.

The proteins having a pilot peptide comprising at least one YxxL motif with SEQ ID NO: 1 include cellular proteins and viral proteins. In particular, these viral proteins are proteins of enveloped viruses, such as transmembrane glycoproteins of enveloped viruses, or herpesvirus proteins, e.g., the LMP2-A protein of the Epstein-Barr virus which comprises at least two YxxL motifs with SEQ ID NO: 1.

In one embodiment, the pilot peptide is that of a transmembrane glycoprotein of a retrovirus. In one embodiment, the pilot peptide may be that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In one embodiment, the pilot peptide comprises one of the following amino acid sequences:

SEQ ID NO: 11:
PxxPxxxxPxxPxSxYxxLxPxxPExYxxLxPxxPDYxxL;
SEQ ID NO: 12:
PxxPxₙPxxPxₙSxYxxLxₙPxxPExₙYxxLxₙPxxPDYxxL;
SEQ ID NO: 13:
PxxPxxxxPxxPxSxYxxLxPxxPExYxxLxPxxPDYxxLxxxx;
and
SEQ ID NO: 14:
PxxPxₙPxxPxₙSxYxxLxₙPxxPExₙYxxLxₙPxxPDYxxLxxxx;

in which “x” and “xₙ”, respectively, represent any amino acid residue and any one or several amino acid residue(s).

In one embodiment, the pilot peptide comprises one of the following amino acid sequences:

SEQ ID NO: 15:
PxxPxxxxxxxxxxxxYxxL;
SEQ ID NO: 16:
PxxPxxxxxxxxxxxDYxxL;
SEQ ID NO: 17:
PxxPxxYxxxxxxxxxYxxL;
SEQ ID NO: 18:
PxxPxxYxxxxxxxxDYxxL;
SEQ ID NO: 19:
PxxPExYxxLxPxxPDYxxL;
SEQ ID NO: 20:
PxxPxₙYxxL;
SEQ ID NO: 21:
PxxPxₙDYxxL;
SEQ ID NO: 22:
PxxPxₙYXₙYxxL;
SEQ ID NO: 23:
PxxPxₙYXₙDYxxL;
SEQ ID NO: 24:
PxxPEXₙYxxLxₙPxxPDYxxL;
SEQ ID NO: 25:
PxxPxxxxPxxPxxxYxxLxPxxPExYxxLxPxxPDYxxL;
SEQ ID NO: 26:
PxxPxₙPxxPxₙYxxLxₙPxxPExₙYxxLxₙPxxPDYxxL;
SEQ ID NO: 27:
PxxPxxxxPxxPxxxYxxLxPxxPExYxxLxPxxPDYxxLxxxx;
and
SEQ ID NO: 28:
PxxPxₙPxxPxₙYxxLxₙPxxPExₙYxxLxₙPxxPDYxxLxxxx,

in which “x” and “xₙ”, respectively, represent any amino acid residue and any one or several amino acid residue(s).

In particular, “n” may be greater than or equal to 1 and less than 50. “n” may, in particular, have any value between 1 and 20.

In one embodiment, the pilot peptide comprises from 6 to 100 amino acid residues, in particular from 20 to 80, from 30 to 70, or from 40 to 60 amino acid residues, for example 40, 41, 42, 43, 44, 45, 46, 47, 48, 49 or 50 amino acid residues.

In one embodiment, the pilot peptide comprises or consists of an amino acid sequence with SEQ ID NO: 30 or a variant thereof. An exemplary nucleic acid sequence coding for the pilot peptide with SEQ ID NO: 30 comprises or consists of SEQ ID NO: 29 or a variant thereof.

SEQ ID NO: 29
GCGCCCCACTTCCCTGAAATCTCCTTCCCCCCTAAACCCGATTCTGATTA
TCAGGCCTTGCTACCATCCGCGCCAGAGATCTACTCTCACCTCTCCCCCA
CCAAACCCGATTACATCAACCTTCGACCGGCGCCCTAA
SEQ ID NO: 30
APHFPEISFPPKPDSDYQALLPSAPEIYSHLSPTKPDYINLRPAP

In one embodiment, a variant of the amino acid sequence with SEQ ID NO: 30 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 30.

Additionally or alternatively, a variant of the amino acid sequence with SEQ ID NO: 30 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 30.

In one embodiment, a variant of the amino acid sequence with SEQ ID NO: 30 retains at least one, two or three YxxL or DYxxL motif(s) with SEQ ID NO: 1 or SEQ ID NO: 4, respectively; and one, two, three or four PxxP motifs with SEQ ID NO: 8.

In one embodiment, the variant of SEQ ID NO: 30 retains at least three YxxL and/or DyxxL motifs with SEQ ID NO: 1 and SEQ ID NO: 4, respectively; and at least four PxxP motifs with SEQ ID NO: 8; wherein “x” represents any amino acid residue.

In one embodiment, a variant of the amino acid sequence with SEQ ID NO: 30 retains three YxxL and/or DYxxL motifs with SEQ ID NO: 1 or SEQ ID NO: 4, respectively; and four PxxP motifs with SEQ ID NO: 8.

In one embodiment, the chimeric polypeptide further comprises at least one linker.

In one embodiment, the at least one linker connects the amino acid sequence of adiponectin and the amino acid sequence of the transmembrane domain.

In one embodiment, the at least one linker is not cleavable. In one embodiment, the at least one linker is cleavable.

In one embodiment, the at least one linker is a Gly-Ser linker. Examples of Gly/Ser linkers include, but are not limited to, GS linkers, G₂S linkers, G₃S linkers, G₄S linkers, including repeats and combination thereof.

In one embodiment, the at least one linker comprises or consists of a (GGGS)ₙ sequence (SEQ ID NO: 38), wherein n is a positive integer ranging from 1 to 10, preferably from 1 to 5.

In one embodiment, the at least one linker comprises or consists of a (GGGSGGGGS)ₙ sequence (SEQ ID NO: 39), wherein n is a positive integer ranging from 1 to 10, preferably from 1 to 5.

In one embodiment, the at least one linker connecting the amino acid sequence of adiponectin and the amino acid sequence of the transmembrane domain comprises the sequence SGGGSGGGGSGGGSGGGGSGGGSGGGGSGGGGS (SEQ ID NO: 53). Said linker may be used in a chimeric polypeptide as described hereinabove, wherein the components i), ii) and, when present, iii) are organized in the chimeric polypeptide from N- to C-terminal (such as construct 1 of FIG. 1).

In one embodiment, the at least one linker connecting the amino acid sequence of adiponectin and the amino acid sequence of the transmembrane domain comprises the sequence GGGSGGGGSGGGSGGGGSGGGSGGGGSGGGSG (SEQ ID NO: 54). Said linker may be used in a chimeric polypeptide as described hereinabove, wherein the components i), ii) and, when present, iii) are organized in the chimeric polypeptide from C- to N-terminal (such as constructs 2, 3, 4, 5 of FIG. 1).

In one embodiment, when the chimeric polypeptide comprising more than one linker, two or more linkers may be identical or different.

In one embodiment, the chimeric polypeptide further comprises an amino acid sequence of a sub-membrane targeting domain.

In one embodiment, a sub-membrane targeting domain is added in a chimeric polypeptide as described hereinabove, wherein the components i), ii) and, when present, iii) are organized in the chimeric polypeptide from C- to N-terminal (such as constructs 2, 3, 4, 5 of FIG. 1).

In one embodiment, the sub-membrane targeting domain is sufficient to allow the chimeric polypeptide to be anchored to the lipid bilayer of cellular or vesicular membranes, preferably via one or more anchoring molecules and/or through interactions such as electrostatic interactions.

Hence, due to its presence in the chimeric polypeptide, the sub-membrane targeting domain allows the chimeric polypeptide, when expressed in a cell, to be anchored to (or anchored in) a cell or vesicular membrane, without the chimeric polypeptide being inserted into said membrane.

In one embodiment, the sub-membrane targeting domain confers to the chimeric polypeptide the property of binding to the inner surface of the cell membrane (i.e., the cytoplasmic side of the cell membrane) and/or to the inner surface of vesicular membranes (i.e., the lumen side of the vesicular membrane).

In one embodiment, the chimeric polypeptide further comprises a sub membrane targeting domain, preferably wherein sub membrane targeting domain is linked to an anchoring molecule.

By “anchoring molecule”, it is meant any molecule capable of being inserted into at least one layer of the lipid bilayer of a cell or vesicular membrane. In particular, the anchoring molecule is a lipid or lipid-containing molecule. The sub-membrane targeting domain is then said to be “lipid-anchored”.

In one embodiment, the anchoring molecule comprises or consists of one or more lipids or lipid-containing molecules, said lipids comprising a hydrophobic carbon chain which allows them to encapsulate in the lipid bilayer of a cell or vesicular membrane.

In one embodiment, the lipids are fatty acids, including, without limitation, myristic acid, palmitic acid, and isoprenoid (such as, e.g., geranyl-geranyl and farnesyl).

In one embodiment, the anchoring molecule is linked to the sub-membrane targeting domain by a covalent bond.

In one embodiment, the anchoring molecule is linked to the sub-membrane targeting domain through a glycine (e.g., in the case of a myristic acid), cysteine or serine amino acid residue of the sub-membrane targeting domain. This link may be through an amide or thioester bond.

In one embodiment, the sub-membrane targeting domain is that of an extrinsic membrane protein or is a variant of the sub-membrane targeting domain of an extrinsic membrane protein.

In one embodiment, the sub-membrane targeting domain comprises or consists of a consensus sequence allowing the attachment (e.g., by acylation or by prenylation) of a fatty acid, and in particular of myristic acid, palmitic acid, or isoprenoid (such as, e.g., geranyl-geranyl and farnesyl).

In one embodiment, the sub-membrane targeting domain comprises or consists of a consensus sequence with SEQ ID NO: 45, as follows:

| (SEQ ID NO: 45) | |
|---|---|
| | (M)G-X₁-X₂-X₃-S/C, |

wherein X₁, X₂, and X₃ independently from each other denote any amino acid residue, and
wherein (M) denotes an initiator methionine which, when located at the N-terminal extremity of the chimeric polypeptide, can be removed in vivo by post-translation processing.

In one embodiment,
X₁ is selected from the group comprising or consisting of C, S and L; and/or
X₂ is selected from the group comprising or consisting of S, I, V, M and L; and/or
X₃ is selected from the group comprising or consisting of K, Q, H, F, C and S.

In one embodiment, when the chimeric polypeptide described herein comprises a sub-membrane targeting domain linked to an anchoring molecule, it is preferably located at the N-terminal position of the chimeric polypeptide.

In one embodiment, the sub-membrane targeting domain may further comprise several basic amino acid residues, in particular several amino acid residues selected from the group comprising or consisting of K, R and H. By “several”, it is meant at least 2, and preferably at least 3 or more. These basic amino acid residues may in particular be involved in interactions with lipids of cell or vesicular membranes, especially with choline and derivative thereof (e.g., with phosphatidylcholine), and thus make it possible to increase the affinity of the sub-membrane targeting domain for these membranes.

In one embodiment, the basic amino acid residues may be located in the consensus sequence with SEQ ID NO: 45 and/or outside this consensus sequence.

Thus, in one embodiment, the sub-membrane targeting domain:
comprises or consists of an amino acid sequence chosen among:

| (SEQ ID NO: 46) | |
|---|---|
| | (M)GXXKS, |
| (SEQ ID NO: 47) | |
| | CKXK, |
| | and |
| (SEQ ID NO: 49) | |
| | CKXKXXXXRRR, |

wherein X denotes any amino acid residue, and
   wherein (M) denotes an initiator methionine which, when located at the N-terminal extremity of the chimeric polypeptide, can be removed in vivo by post-translation processing; or
is a variant of any of these sequences, said variant retaining the ability of the sub-membrane targeting domain to be anchored in the lipid bilayer of a cell or vesicular membrane.

In one embodiment, the sub-membrane targeting domain is derived from a protein of the Src family of proteins. Examples of such proteins include, without limitation, Src, Yes, Lyn, Fyn, Lck, Blk, Fgr, Hck and Yrk proteins (Resh, 1994. Cell. 76 (3): 411-413), and more particularly the N-terminal portion of one of these proteins, such as 15, 16, 17, 18, 19, 20, 21, 22, 23, 24 or 25 N-terminal amino acid residues of one of these proteins.

In one embodiment, the sub-membrane targeting domain is derived from the c-Src or v-Src protein, and preferably from the c-Src.

Alternatively, the sub-membrane targeting domain may be derived from other acylated proteins, such as, e.g., viral capsid proteins, including, without limitation, the human immunodeficiency virus (HIV) MA protein, or filovirus proteins.

In one embodiment, the sub-membrane targeting domain is derived from a Src protein.

In one embodiment, the sub-membrane targeting domain is derived from a Src protein and comprises or consists of one of the following amino acid sequences:

| (SEQ ID NO: 50) | |
|---|---|
| | (M)GSSKSKPKDPSQRRR, |
| (SEQ ID NO: 51) | |
| | (M)GSSKSKPKDPSQRRRKSR |
| (SEQ ID NO: 52) | |
| | (M)GSSKSKPKDPSQRRRKSRGPGG, |

or
a variant of any of these two sequences, said variant retaining the ability of the sub-membrane targeting domain to be anchored in the lipid bilayer of a cell or vesicular membrane;
   wherein (M) denotes an initiator methionine which, when located at the N-terminal extremity of the chimeric polypeptide, can be removed in vivo by post-translation processing.

In one embodiment, the sub-membrane targeting domain is derived from a Src protein and comprises or consists of an amino acid sequence with SEQ ID NO: 51 or a variant thereof.

In one embodiment, a variant of the amino acid sequence with SEQ ID NO: 50, 51 or 52 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 50, 51 or 52.

Additionally or alternatively, a variant of the amino acid sequence with SEQ ID NO: 50, 51 or 52 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 50, 51 or 52.

In one embodiment, the sub-membrane targeting domain is derived from a Src protein and comprises or consists of an amino acid sequence with SEQ ID NO: 50, 51 or 52 or a variant thereof, preferably with SEQ ID NO: 51; and further comprises one or more anchoring molecules as defined above; in particular, comprises a myristic acid (in the form of a myristyl moiety) linked to the glycine residue at position 2.

In one embodiment, when the chimeric polypeptide comprises a sub-membrane targeting domain, this sub-membrane targeting domain may be linked to the remaining portions of the chimeric polypeptides thought at least one linker.

In one embodiment, the chimeric polypeptide comprises or consists of, from N-terminal to C-terminal:
an amino acid sequence of adiponectin or a variant thereof, as defined hereinabove;
an amino acid sequence of a transmembrane domain of a transmembrane protein, as defined hereinabove;
an amino acid sequence of a pilot peptide, as defined hereinabove.

Optionally, one or several linker(s) may be added between the components of the chimeric polypeptide of the present invention.

In one embodiment, the chimeric polypeptide comprises or consists of, from N-terminal to C-terminal:
an amino acid sequence of adiponectin or a variant thereof, as defined hereinabove;
optionally, a linker;
an amino acid sequence of a transmembrane domain of a transmembrane protein, as defined hereinabove;
optionally, a linker; and
an amino acid sequence of a pilot peptide, as defined hereinabove.

An example of such construct is shown in FIG. 1, as “Construct 1”.

In one embodiment, the chimeric polypeptide comprises or consists of, from N-terminal to C-terminal:
an amino acid sequence of human wild-type adiponectin or a variant thereof, as defined hereinabove,
optionally, a linker,
an amino acid sequence of a transmembrane domain of CD8, as defined hereinabove,
optionally, a linker, and
an amino acid sequence of a pilot peptide, as defined hereinabove; preferably the pilot peptide comprises at least one YxxL and/or DYxxL motif with SEQ ID NO: 1 or SEQ ID NO: 4, respectively; and at least one PxxP motif with SEQ ID NO: 8.

In one embodiment, the chimeric polypeptide comprises or consists of, from N-terminal to C-terminal:
an amino acid sequence of human wild-type adiponectin or a variant thereof, as defined hereinabove,
optionally, a linker,
an amino acid sequence of a transmembrane domain of CD8, as defined hereinabove,
optionally, a linker, and
an amino acid sequence of a pilot peptide, as defined hereinabove; preferably the pilot peptide comprises three YxxL and/or DYxxL motifs with SEQ ID NO: 1 or SEQ ID NO: 4, respectively; and four PxxP motifs with SEQ ID NO: 8.

In one embodiment, the chimeric polypeptide comprises or consists of, from N-terminal to C-terminal:
an amino acid sequence of human wild-type adiponectin with SEQ ID NO: 33 or a variant thereof,
optionally, a linker,
an amino acid sequence of a transmembrane domain of CD8 with SEQ ID NO: 37 or a variant thereof,
optionally, a linker, and
an amino acid sequence of a pilot peptide with SEQ ID NO: 30 or a variant thereof.

In one embodiment, the chimeric polypeptide comprises or consists of the amino acid sequence with SEQ ID NO: 40, or a variant thereof.

SEQ ID NO: 40
MLLLGAVLLLLALPGHDQETTTQGPGVLLPLPKGACTGWMAGIPGHPGHN
GAPGRDGRDGTPGEKGEKGDPGLIGPKGDIGETGVPGAEGPRGFPGIQGR
KGEPGEGAYVYRSAFSVGLETYVTIPNMPIRFTKIFYNQQNHYDGSTGKF
HCNIPGLYYFAYHITVYMKDVKVSLFKKDKAMLFTYDQYQENNVDQASGS
VLLHLEVGDQVWLQVYGEGERNGLYADNDNDSTFTGFLLYHDTNSGGGSG
GGGSGGGSGGGGSGGGSGGGGSGGGGSDIYIWAPLAGICVALLLSLIITL
ICYHRSRGAPHFPEISFPPKPDSDYQALLPSAPEIYSHLSPTKPDYINLR
PAP

In one embodiment, a variant of the amino acid sequence with SEQ ID NO: 40 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 40.

Additionally or alternatively, a variant of the amino acid sequence with SEQ ID NO: 40 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 40.

In one embodiment, the chimeric polypeptide comprises or consists of, from N-terminal to C-terminal:
optionally, an amino acid sequence of a pilot peptide, as defined hereinabove,
an amino acid sequence of a transmembrane domain of a transmembrane protein, as defined hereinabove,
an amino acid sequence of adiponectin or a variant thereof, as defined hereinabove.

Optionally, one or several linker(s) may be added between the components of the chimeric polypeptide of the present invention.

In one embodiment, the chimeric polypeptide comprises or consists of, from N-terminal to C-terminal:
optionally, an amino acid sequence of a pilot peptide, as defined hereinabove,
optionally, a linker,
an amino acid sequence of a transmembrane domain of a transmembrane protein, as defined hereinabove,
optionally, a linker, and
an amino acid sequence of adiponectin or a variant thereof, as defined hereinabove.

An example of such constructs is shown in FIG. 1, as “Construct 2” and “Construct 3”.

In one embodiment, the chimeric polypeptide comprises or consists of, from N-terminal to C-terminal:
optionally, an amino acid sequence of a pilot peptide, as defined hereinabove; preferably the pilot peptide comprises at least one YxxL and/or DYxxL motif with SEQ ID NO: 1 or SEQ ID NO: 4, respectively; and at least one PxxP motif with SEQ ID NO: 8,
optionally, a linker,
an amino acid sequence of a transmembrane domain of CD40L, as defined hereinabove,
optionally, a linker, and
an amino acid sequence of human wild-type adiponectin or a variant thereof, as defined hereinabove.

In one embodiment, the chimeric polypeptide comprises or consists of, from N-terminal to C-terminal:
an amino acid sequence of a pilot peptide, as defined hereinabove; preferably the pilot peptide comprises at least one YxxL and/or DYxxL motif with SEQ ID NO: 1 or SEQ ID NO: 4, respectively; and at least one PxxP motif with SEQ ID NO: 8,
an amino acid sequence of a transmembrane domain of CD40L, as defined hereinabove,
an amino acid sequence of human wild-type adiponectin or a variant thereof, as defined hereinabove.

Optionally, one or several linker(s) may be added between the components of the chimeric polypeptide of the present invention.

In one embodiment, the chimeric polypeptide comprises or consists of, from N-terminal to C-terminal:
optionally, an amino acid sequence of a pilot peptide, as defined hereinabove; preferably the pilot peptide comprises three YxxL and/or DYxxL motifs with SEQ ID NO: 1 or SEQ ID NO: 4, respectively; and four PxxP motifs with SEQ ID NO: 8,
optionally, a linker,
an amino acid sequence of a transmembrane domain of CD40L, as defined hereinabove,
optionally, a linker, and
an amino acid sequence of human wild-type adiponectin or a variant thereof, as defined hereinabove.

In one embodiment, the chimeric polypeptide comprises or consists of, from N-terminal to C-terminal:
an amino acid sequence of a pilot peptide, as defined hereinabove; preferably the pilot peptide comprises three YxxL and/or DYxxL motifs with SEQ ID NO: 1 or SEQ ID NO: 4, respectively; and four PxxP motifs with SEQ ID NO: 8,
an amino acid sequence of a transmembrane domain of CD40L, as defined hereinabove,
an amino acid sequence of human wild-type adiponectin or a variant thereof, as defined hereinabove.

Optionally, one or several linker(s) may be added between the components of the chimeric polypeptide of the present invention.

In one embodiment, the chimeric polypeptide comprises or consists of, from N-terminal to C-terminal:
an amino acid sequence of a pilot peptide with SEQ ID NO: 30 or a variant thereof,
an amino acid sequence of a transmembrane domain of CD40L, as defined hereinabove,
an amino acid sequence of human wild-type adiponectin or a variant thereof, as defined hereinabove.

Optionally, one or several linker(s) may be added between the components of the chimeric polypeptide of the present invention.

In one embodiment, the chimeric polypeptide comprises or consists of, from N-terminal to C-terminal:
optionally, an amino acid sequence of a pilot peptide with SEQ ID NO: 30 or a variant thereof,
optionally, a linker,
an amino acid sequence of a transmembrane domain of CD40L with SEQ ID NO: 35 or a variant thereof,
optionally, a linker, and
an amino acid sequence of human wild-type adiponectin with SEQ ID NO: 31 or a variant thereof.

In one embodiment, the chimeric polypeptide comprises or consists of, from N-terminal to C-terminal:
an amino acid sequence of a pilot peptide with SEQ ID NO: 30 or a variant thereof,
an amino acid sequence of a transmembrane domain of CD40L with SEQ ID NO: 35 or a variant thereof,
an amino acid sequence of human wild-type adiponectin with SEQ ID NO: 31 or a variant thereof.

Optionally, one or several linker(s) may be added between the components of the chimeric polypeptide of the present invention.

In one embodiment, the chimeric polypeptide comprises or consists of the amino acid sequence with SEQ ID NO: 41, or a variant thereof.

SEQ ID NO: 41
MSRGSMIETYNQTSPRSAATGLPISMKIFMYLLTVFLITQMIGSALFAVY
LHRRLDKGGGSGGGGSGGGSGGGGSGGGSGGGGSGGGSGETTTQGPGVLL
PLPKGACTGWMAGIPGHPGHNGAPGRDGRDGTPGEKGEKGDPGLIGPKGD
IGETGVPGAEGPRGFPGIQGRKGEPGEGAYVYRSAFSVGLETYVTIPNMP
IRFTKIFYNQQNHYDGSTGKFHCNIPGLYYFAYHITVYMKDVKVSLFKKD
KAMLFTYDQYQENNVDQASGSVLLHLEVGDQVWLQVYGEGERNGLYADND
NDSTFTGFLLYHDTN

In one embodiment where the pilot peptide is not optional in the above embodiments, the chimeric polypeptide comprises or consists of the amino acid sequence with SEQ ID NO: 42, or a variant thereof.

SEQ ID NO: 42
MSRGGGAPHFPEISFPPKPDSDYQALLPSAPEIYSHLSPTKPDYINLRPA
PGGSRGSMIETYNQTSPRSAATGLPISMKIFMYLLTVFLITQMIGSALFA
VYLHRRLDKGGGSGGGGSGGGSGGGGSGGGSGGGGSGGGSGETTTQGPGV
LLPLPKGACTGWMAGIPGHPGHNGAPGRDGRDGTPGEKGEKGDPGLIGPK
GDIGETGVPGAEGPRGFPGIQGRKGEPGEGAYVYRSAFSVGLETYVTIPN
MPIRFTKIFYNQQNHYDGSTGKFHCNIPGLYYFAYHITVYMKDVKVSLFK
KDKAMLFTYDQYQENNVDQASGSVLLHLEVGDQVWLQVYGEGERNGLYAD
NDNDSTFTGFLLYHDTN

In one embodiment, a variant of the amino acid sequence with SEQ ID NO: 41 or SEQ ID NO: 42 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 41 or SEQ ID NO: 42, respectively.

Additionally or alternatively, a variant of the amino acid sequence with SEQ ID NO: 41 or SEQ ID NO: 42 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 41 or SEQ ID NO: 42, respectively.

In one embodiment, the chimeric polypeptide comprises or consists of, from N-terminal to C-terminal:
an amino acid sequence of a sub-membrane targeting domain, as defined hereinabove; preferably wherein said sub-membrane targeting domain is linked to an anchoring molecule,
optionally, an amino acid sequence of a pilot peptide, as defined hereinabove,
an amino acid sequence of a transmembrane domain of a transmembrane protein, as defined hereinabove,
an amino acid sequence of adiponectin or a variant thereof, as defined hereinabove.

Optionally, one or several linker(s) may be added between the components of the chimeric polypeptide of the present invention.

In one embodiment, the chimeric polypeptide comprises or consists of, from N-terminal to C-terminal:
an amino acid sequence of a sub-membrane targeting domain, as defined hereinabove; preferably wherein said sub-membrane targeting domain is linked to an anchoring molecule,
optionally, a linker,
optionally, an amino acid sequence of a pilot peptide, as defined hereinabove,
optionally, a linker,
an amino acid sequence of a transmembrane domain of a transmembrane protein, as defined hereinabove,
optionally, a linker, and
an amino acid sequence of adiponectin or a variant thereof, as defined hereinabove.

An example of such constructs is shown in FIG. 1, as “Construct 4” and “Construct 5”.

In one embodiment, the chimeric polypeptide comprises or consists of, from N-terminal to C-terminal:
an amino acid sequence of a sub-membrane targeting domain with sequence (M) G-X₁-X₂-X₃-S/C (SEQ ID NO: 45), as defined hereinabove; preferably wherein said sub-membrane targeting domain is linked to a fatty acid,
optionally, a linker,
optionally, an amino acid sequence of a pilot peptide, as defined hereinabove; preferably the pilot peptide comprises at least one YxxL and/or DYxxL motif with SEQ ID NO: 1 or SEQ ID NO: 4, respectively; and at least one PxxP motif with SEQ ID NO: 8,
optionally, a linker,
an amino acid sequence of a transmembrane domain of CD40L, as defined hereinabove,
optionally, a linker, and
an amino acid sequence of human wild-type adiponectin or a variant thereof, as defined hereinabove.

In one embodiment, the chimeric polypeptide comprises or consists of, from N-terminal to C-terminal:
an amino acid sequence of a sub-membrane targeting domain with sequence (M) G-X₁-X₂-X₃-S/C (SEQ ID NO: 45), as defined hereinabove; preferably wherein said sub-membrane targeting domain is linked to a fatty acid,
optionally, a linker,
optionally, an amino acid sequence of a pilot peptide, as defined hereinabove; preferably the pilot peptide comprises three YxxL and/or DYxxL motifs with SEQ ID NO: 1 or SEQ ID NO: 4, respectively; and four PxxP motifs with SEQ ID NO: 8,
optionally, a linker,
an amino acid sequence of a transmembrane domain of CD40L, as defined hereinabove,
optionally, a linker, and
an amino acid sequence of human wild-type adiponectin or a variant thereof, as defined hereinabove.

In one embodiment, the chimeric polypeptide comprises or consists of, from N-terminal to C-terminal:
an amino acid sequence of a sub-membrane targeting domain with SEQ ID NO: 51 preferably wherein said sub-membrane targeting domain is linked to a fatty acid, preferably a myristic acid,
optionally, a linker,
optionally, an amino acid sequence of a pilot peptide with SEQ ID NO: 30 or a variant thereof,
optionally, a linker,
an amino acid sequence of a transmembrane domain of CD40L with SEQ ID NO: 35 or a variant thereof,
optionally, a linker, and
an amino acid sequence of human wild-type adiponectin with SEQ ID NO: 31 or a variant thereof.

In one embodiment, the chimeric polypeptide comprises or consists of the amino acid sequence with SEQ ID NO: 43, or a variant thereof.

SEQ ID NO: 43
MGSSKSKPKDPSQRRRKSRGPGGGSGGGSGGGSGGGSSRGSMIETYNQTS
PRSAATGLPISMKIFMYLLTVFLITQMIGSALFAVYLHRRLDKGGGSGGG
GSGGGSGGGGSGGGSGGGGSGGGSGETTTQGPGVLLPLPKGACTGWMAGI
PGHPGHNGAPGRDGRDGTPGEKGEKGDPGLIGPKGDIGETGVPGAEGPRG
FPGIQGRKGEPGEGAYVYRSAFSVGLETYVTIPNMPIRFTKIFYNQQNHY
DGSTGKFHCNIPGLYYFAYHITVYMKDVKVSLFKKDKAMLFTYDQYQENN
VDQASGSVLLHLEVGDQVWLQVYGEGERNGLYADNDNDSTFTGFLLYHDT
N

In one embodiment where the pilot peptide is not optional in the above embodiments, the chimeric polypeptide comprises or consists of the amino acid sequence with SEQ ID NO: 44, or a variant thereof.

SEQ ID NO: 44
MGSSKSKPKDPSQRRRKSRGPGGGSGGGSGGGSGGGSSRGGGAPHFPEIS
FPPKPDSDYQALLPSAPEIYSHLSPTKPDYINLRPAPGGSRGSMIETYNQ
TSPRSAATGLPISMKIFMYLLTVFLITQMIGSALFAVYLHRRLDKGGGSG
GGGSGGGSGGGGSGGGSGGGGSGGGSGETTTQGPGVLLPLPKGACTGWMA
GIPGHPGHNGAPGRDGRDGTPGEKGEKGDPGLIGPKGDIGETGVPGAEGP
RGFPGIQGRKGEPGEGAYVYRSAFSVGLETYVTIPNMPIRFTKIFYNQQN
HYDGSTGKFHCNIPGLYYFAYHITVYMKDVKVSLFKKDKAMLFTYDQYQE
NNVDQASGSVLLHLEVGDQVWLQVYGEGERNGLYADNDNDSTFTGFLLYH
DTN

In one embodiment, a variant of the amino acid sequence with SEQ ID NO: 43 or SEQ ID NO: 44 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 43 or SEQ ID NO: 44, respectively.

Additionally or alternatively, a variant of the amino acid sequence with SEQ ID NO: 43 or SEQ ID NO: 44 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 43 or SEQ ID NO: 44, respectively.

The present invention further relates to a nucleic acid encoding the chimeric polypeptide, as defined hereinabove.

In one embodiment, the nucleic acid is comprised in a nucleic acid vector, such as a nucleic acid expression vector.

In one embodiment, the nucleic acid is comprised in a nucleic acid vector such as a nucleic acid expression vector, and is operably linked to regulatory elements.

Examples of regulatory elements include, but are not limited to, promoters, Kozak consensus initiation sequence, polyadenylation signal, termination sequence (i.e., stop codon), and the like. In particular, the regulatory elements are suitable for expression of the nucleic acid in a cell, such as a bacterium, a yeast, an insect cell, a mammalian cell, or a human cell.

The present invention further relates to an extracellular vesicle (EV) comprising a chimeric polypeptide, as defined hereinabove.

In one embodiment, the extracellular vesicle harbors at its outer surface the adiponectin comprised in the chimeric polypeptide. In one embodiment, the transmembrane domain of the chimeric polypeptide is anchored in the extracellular vesicle lipid bilayer.

As used herein, the expression “harbors at its outer surface” means that the adiponectin comprised in the chimeric polypeptide is exposed, partially or completely, outside the extracellular vesicle. This configuration enables the oligomerization of the adiponectin comprised in the chimeric polypeptide, either with other adiponectin of neighboring chimeric polypeptides in the same extracellular vesicle, or with soluble adiponectin.

In one embodiment, the extracellular vesicle is a small extracellular vesicle.

In one embodiment, the extracellular vesicle is an exosome.

In one embodiment, exosomes have a diameter ranging from about 30 nm to about 150 nm, preferably from about 30 nm to about 120 nm, more preferably from about 40 nm to about 80 nm. In one embodiment, exosomes have a diameter ranging from about 30 nm to about 120 nm.

A further object of the present invention is a population of extracellular vesicles, as defined hereinabove.

In one embodiment, the population of extracellular vesicles is monodisperse in aqueous solutions, preferably in a NaCl 0.9% aqueous solution and/or in PBS.

By “monodisperse”, it is meant that the extracellular vesicles in the population of extracellular vesicles are substantially uniform in size. By “substantially uniform”, it is meant that the extracellular vesicles have a narrow distribution of sizes around an average size. In one embodiment, the extracellular vesicles in NaCl 0.9% aqueous solution and/or in PBS have sizes exhibiting a standard deviation of less than 100% with respect to their average size, such as less than 75%, 50%, 40%, 30%, 20%, 10%, or less than 5%.

In one embodiment, the population of extracellular vesicles further comprise soluble adiponectin, i.e., adiponectin proteins that are in free form; in other words, not comprised in a chimeric polypeptide according to the invention.

In one embodiment, the soluble adiponectin is a wild-type adiponectin. In one embodiment, the soluble adiponectin is a mutant adiponectin.

In one embodiment, the soluble adiponectin is a the wild-type human adiponectin. In one embodiment, the wild-type human adiponectin comprises or consists of the amino acid sequence with SEQ ID NO: 33, or a variant thereof. In one embodiment, the wild-type human adiponectin comprises or consists of the amino acid sequence with SEQ ID NO: 31, or a variant thereof.

A further object of the present invention is a method of obtaining an extracellular vesicle or a population of extracellular vesicles comprising a chimeric polypeptide, as defined hereinabove.

General means and methods for obtaining extracellular vesicles or a population of extracellular vesicles are well known in the art. See, e.g., Whitford & Guterstam, 2019. Future Med Chem. 11 (10): 1225-1236; Taylor & Shah, 2015. Methods. 87:3-10; Desplantes et al., 2017. Sci Rep. 7 (1): 1032.

In one embodiment, the method for obtaining an extracellular vesicle or a population of extracellular vesicles comprises a step of producing the extracellular vesicle or the population of extracellular vesicles, as defined hereinabove.

In one embodiment, this step of producing the extracellular vesicle or the population of extracellular vesicles comprises transfecting cells with a nucleic acid encoding the chimeric polypeptide, as defined hereinabove.

In one embodiment, the cells are HEK293T cells or cells from a derivative cell line. In one embodiment, the cells are adipocytes. In one embodiment, the cells are immune cells, including, but not limited to, mastocytes, lymphocytes (such as, e.g., T-cells or B-cells), and dendritic cells. In one embodiment, the cells are stem cells, including, but not limited to, embryonic stem cells, adult stem cells (such as, e.g., hematopoietic stem cells, mammary stem cells, intestinal stem cells, mesenchymal stem cells, endothelial stem cells, neural stem cells, olfactory adult stem cells, or neural crest stem cells), cancer stem cells, induced pluripotent stem cells (iPSC) and induced stem cells (iSC).

In one embodiment, the method for obtaining an extracellular vesicle or a population of extracellular vesicles further comprises a step of culturing the transfected cells for a time sufficient to allow extracellular vesicle production, preferably in a medium devoid of extracellular vesicles (i.e., a serum-free medium, a medium supplemented with extracellular vesicle-depleted serum, or a medium supplemented with extracellular vesicle-depleted platelet lysate).

In one embodiment, the method for obtaining an extracellular vesicle or a population of extracellular vesicles further comprises a step of purifying said extracellular vesicle or population of extracellular vesicles.

In one embodiment, the step of purifying said extracellular vesicle or population of extracellular vesicles comprises clarification (such as, e.g., by centrifugation or by depth-filtration), filtration, ultra-filtration, diafiltration, size-exclusion purification and/or ion exchange chromatography of the transfected cell culture supernatant. Other methods to purify extracellular vesicle or population of extracellular vesicles include, without limitation, ultra-centrifugation, tangential flow filtration (TFF) and BE-SEC chromatography.

In one embodiment, the extracellular vesicle or population of extracellular vesicles of the present invention is purified. Thus, the present invention also relates to a purified extracellular vesicle or population of extracellular vesicles.

Methods for purification are well-known by the skilled artisan in the art and includes, without limitation, the methods of purification as described hereinabove.

In one embodiment, the extracellular vesicle or population of extracellular vesicles of the present invention is purified by ultra-centrifugation to obtain semi-purified extracellular vesicle or population of extracellular vesicles. Thus, the present invention also relates to a semi-purified extracellular vesicle or population of extracellular vesicles.

As used herein, a semi-purified extracellular vesicle or population of extracellular vesicles comprises the extracellular vesicle or the population of extracellular vesicles, the proteins anchored in the membrane of the extracellular vesicle or the population of extracellular vesicles or tightly associated with the extracellular vesicle or the population of extracellular vesicles, as well as the crown of proteins associated with the extracellular vesicle or the population of extracellular vesicles (see FIG. 2B).

In one embodiment, the extracellular vesicle or population of extracellular vesicles of the present invention is purified by tangential flow filtration and chromatography, in particular SEC and BE-SEC chromatography, to obtain ultra-purified extracellular vesicle or population of extracellular vesicles. Thus, the present invention also relates to an ultra-purified extracellular vesicle or population of extracellular vesicles.

As used herein, an ultra-purified extracellular vesicle or population of extracellular vesicles comprises the extracellular vesicle or the population of extracellular vesicles, and the proteins anchored in the membrane of the extracellular vesicle or the population of extracellular vesicles or tightly associated with the extracellular vesicle or the population of extracellular vesicles (see FIG. 2C).

An example of the production of extracellular vesicles in HEK293T cells is provided in the Example section below.

The present invention further relates to a composition comprising, consisting essentially of or consisting of the chimeric polypeptide, the nucleic acid, the extracellular vesicle or the population of extracellular vesicles, as defined hereinabove.

As used herein, “consisting essentially of”, with reference to a composition, means that the chimeric polypeptide, the nucleic acid, the extracellular vesicle or the population of extracellular vesicles is the only one therapeutic agent or agent with a biologic activity within said composition.

In one embodiment, the composition further comprises soluble adiponectin, i.e., adiponectin proteins that are in free form, as already defined above.

The present invention further relates to a pharmaceutical composition comprising, consisting essentially of or consisting of the chimeric polypeptide, the nucleic acid, the extracellular vesicle or the population of extracellular vesicles, as defined hereinabove, and at least one pharmaceutically acceptable excipient.

The term “pharmaceutically acceptable excipient” includes any and all solvents, diluents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of vegetable oil saturated fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., sodium carboxymethylcellulose), polyethylene glycol, polyacrylates, waxes, polyethylene-polyoxypropylene-block polymers, polyethylene glycol and wool fat.

In one embodiment, the pharmaceutical composition further comprises soluble adiponectin, i.e., adiponectin proteins that are in free form, as already defined above.

The present invention further relates to a medicament comprising, consisting essentially of or consisting of the chimeric polypeptide, the nucleic acid, the extracellular vesicle or the population of extracellular vesicles, as defined hereinabove.

In one embodiment, the medicament further comprises soluble adiponectin, i.e., adiponectin proteins that are in free form, as already defined above.

In one embodiment, the composition, the pharmaceutical composition or the medicament comprises a purified extracellular vesicle or population of extracellular vesicles as defined hereinabove.

The present invention further relates to a kit-of-parts comprising, in a first part, the extracellular vesicle or the population of extracellular vesicle, as defined hereinabove; and, in a second part, soluble adiponectin, i.e., adiponectin proteins that are in free form, as already defined above.

In one embodiment, the two parts of the kit-of-parts are intended for simultaneous use, or for sequential use in any order.

The present invention further relates to the chimeric polypeptide, the nucleic acid, the extracellular vesicle, the population of extracellular vesicles, the composition, the pharmaceutical composition, the medicament or the kit-of-parts, as defined hereinabove, for use as a drug or medicament.

The present invention further relates to the chimeric polypeptide, the nucleic acid, the extracellular vesicle, the population of extracellular vesicles, the composition, the pharmaceutical composition, the medicament or the kit-of-parts, as defined hereinabove, for use in treating a disease, disorder or condition in a subject in need thereof.

The present invention further relates to the use of the chimeric polypeptide, the nucleic acid, the extracellular vesicle, the population of extracellular vesicles, the composition, the pharmaceutical composition, the medicament or the kit-of-parts, as defined hereinabove, in the manufacture of a medicament for treating a disease, disorder or condition in a subject in need thereof.

The present invention further relates to a method for treating a disease, disorder or condition in a subject in need thereof, comprising or consisting of administering the chimeric polypeptide, the nucleic acid, the extracellular vesicle, the population of extracellular vesicles, the composition, the pharmaceutical composition, the medicament or the kit-of-parts, as defined hereinabove, to the subject.

In one embodiment, the disease, disorder or condition is selected from the group comprising or consisting of obesity, insulin resistance, diseases related to insulin resistance or deficiency, hypertension, dyslipidemia, hyperuricemia, atherosclerosis (including coronary artery disease, stroke and peripheral artery disease), fibrosis, inflammatory pulmonary diseases, nephrotic disease, sleep apnea, dry eye diseases, inflammatory ocular diseases, gastritis and gastro-esophageal reflux disease, inflammatory bowel disease, pancreatitis, osteoporosis, and inflammatory bone and joint diseases. In one embodiment, the disease, disorder or condition is diabetes.

Examples of diseases related to insulin resistance or deficiency include, but are not limited to, type 2 diabetes, metabolic syndrome, cardiovascular disease, non-alcoholic fatty liver disease, polycystic ovary syndrome, Alzheimer's disease, and cancer (in particular, endometrial cancer, postmenopausal breast cancer, leukemia, colon cancer, gastric cancer, and prostate cancer). In one embodiment, the disease is type 2 diabetes.

Examples of inflammatory pulmonary diseases include, but are not limited to, asthma, allergic asthma, emphysema, chronic obstructive pulmonary disease (COPD), acute respiratory distress syndrome, bronchitis, pneumonia, cystic fibrosis, pulmonary fibrosis, and pulmonary sarcoidosis.

Examples of dry eye diseases include, but are not limited to, hypolacrimation, tear deficiency, xerophthalmia, Sjogren's syndrome dry eye, non-Sjogren's syndrome dry eye, keratoconjuctivitis sicca, aqueous tear-deficiency dry eye, evaporative dry eye, Stevens-Johnson syndrome, ocular pemphigoid blepharitis marginal, allergic conjunctivitis-associated dry eye, post-viral conjunctivitis-associated dry eye, post-cataract surgery-associated dry eye, visual display terminals operation-associated dry eye, and contact lens wearing-associated dry eye.

Examples of inflammatory ocular diseases include, but are not limited to, uveitis, scleritis, post-eye surgery inflammation, corneal transplantation, corneal wound healing, conjunctivitis, retinal disease, glaucoma, and ocular hypertension.

Examples of inflammatory bone and joint diseases include, but are not limited to, osteitis fibrosa cystica, osteomyelitis, sesamoiditis, Brodie abscess, periostitis, costochondritis, and polychondritis.

In one embodiment, the disease, disorder or condition is hypoadiponectinemia, or is associated with hypoadiponectinemia.

As used herein, “hypoadiponectinemia” refers to a reduced level of adiponectin in the bloodstream, as compared to standard levels. Methods for measuring the level of adiponectin in the bloodstream are well-know by the skilled artisan and include, without limitation, enzyme-linked immunosorbent assay (ELISA), AlphaLISA immunoassays, and turbidimetric immunoassay (such as latex particle-enhanced turbidimetric immunoassay).

In one embodiment, the disease, disorder or condition is obesity. In one embodiment, the disease, disorder or condition is insulin resistance. In one embodiment, the disease, disorder or condition is a disease related to insulin resistance or deficiency, such as type 2 diabetes, metabolic syndrome, cardiovascular disease, non-alcoholic fatty liver disease, polycystic ovary syndrome, Alzheimer's disease, and cancer; in particular type 2 diabetes and metabolic syndrome. In one embodiment, the disease, disorder or condition is type 2 diabetes.

The present invention also relates to an extracellular vesicle harboring adiponectin exposed at its outer surface, or a population thereof, for use in treating a disease, disorder or condition selected from the group comprising obesity, insulin resistance, diseases related to insulin resistance or deficiency, hypertension, dyslipidemia, hyperuricemia, atherosclerosis (including coronary artery disease, stroke and peripheral artery disease), fibrosis, inflammatory pulmonary diseases, nephrotic disease, sleep apnea, dry eye diseases, inflammatory ocular diseases, gastritis and gastro-esophageal reflux disease, inflammatory bowel disease, pancreatitis, osteoporosis, and inflammatory bone and joint diseases. In one embodiment, the disease, disorder or condition is diabetes.

In one embodiment, the extracellular vesicle is partially or totally coated with recombinant adiponectin.

By “recombinant adiponectin”, it is meant exogenous adiponectin which is not endogenously produced by a cell. Extracellular vesicles partially or totally coated with recombinant adiponectin can be obtained by contacting extracellular vesicles with adiponectin, either in cellulo (e.g., by transfecting an extracellular vesicle-producing cell with a nucleic acid encoding adiponectin, thereby having the cell produce exogenous adiponectin) or ex cellulo (e.g., by providing adiponectin in protein form, previously produced in a suitable recombinant expression system and further purified).

By “coated”, it is implied that adiponectin is exposed at the outer surface of the extracellular vesicle, to which it is bound through any suitable type of interaction with external components of the vesicle (such as, without limitation, electrostatic interactions, protein-protein interactions, protein-lipid interactions, etc.).

In one embodiment, the extracellular vesicle is partially or completely coated with recombinant adiponectin fused to lactadherin, in particular to a functional C1 and/or C2 domain of lactadherin, as described in International application WO2003016522, the relevant content of which is incorporated herein by reference.

In one embodiment, the chimeric polypeptide, the nucleic acid, the extracellular vesicle, the population of extracellular vesicles, the composition, the pharmaceutical composition, the medicament or the components of the kit-of-parts, as defined hereinabove, is/are formulated for administration to a subject in need thereof.

In one embodiment, administration to a subject can be performed parenterally, by inhalation spray, rectally, nasally, or via an implanted reservoir. The term “administration” includes, inter alia, subcutaneous, intravenous, intramuscular, intra-articular, intra-synovial, intrasternal, intrathecal, intrahepatic, intralesional and intracranial injection or infusion techniques.

In one embodiment, the chimeric polypeptide, the nucleic acid, the extracellular vesicle, the population of extracellular vesicles, the composition, the pharmaceutical composition, the medicament or the components of the kit-of-parts, as defined hereinabove, is/are to be administered to a subject in need thereof in a therapeutically effective amount.

It will be however understood that the total daily usage of the chimeric polypeptide, the nucleic acid, the extracellular vesicle, the population of extracellular vesicles, the composition, the pharmaceutical composition, the medicament or the components of the kit-of-parts, as defined hereinabove, will be decided by the attending physician within the scope of sound medical judgment.

In particular, the specific therapeutically effective dose level for any particular patient will depend upon a variety of factors including the disease being treated and the severity of the disease; activity of the chimeric polypeptide, the nucleic acid, the extracellular vesicle, the population of extracellular vesicles, the composition, the pharmaceutical composition, the medicament or the components of the kit-of-parts, as defined hereinabove, employed; the age, body weight, general health, sex and diet of the subject; the time of administration, route of administration, and rate of excretion of the chimeric polypeptide, the nucleic acid, the extracellular vesicle, the population of extracellular vesicles, the composition, the pharmaceutical composition, the medicament or the components of the kit-of-parts, as defined hereinabove, employed; the duration of the treatment; drugs used in combination or coincidental with the chimeric polypeptide, the nucleic acid, the extracellular vesicle, the population of extracellular vesicles, the composition, the pharmaceutical composition, the medicament or the components of the kit-of-parts, as defined hereinabove, employed; and like factors well known in the medical arts. The total dose required for each treatment may be administered by multiple doses or in a single dose.

The present invention also relates to a method of diagnosing obesity, insulin resistance, or a disease related to insulin resistance or deficiency in a subject, comprising the steps of:
a) measuring the level or amount of adiponectin-associated small extracellular vesicles in a sample previously retrieved from the subject;
b) comparing the level or amount determined at step a) with a reference level or amount of adiponectin-associated small extracellular vesicles, wherein the reference level or amount was pre-determined in a sample from a reference subject known not to be suffering from obesity or insulin resistance, or in a pool of samples from more than one reference subjects, all known not to be suffering from obesity or insulin resistance;
c) concluding that the subject is affected with obesity, insulin resistance, or a disease related to insulin resistance or deficiency, when the level or amount determined at step a) is lower than the reference level or amount.

The present invention also relates to the in vitro or in vivo use of the chimeric polypeptide, the nucleic acid, the extracellular vesicle, the population of extracellular vesicles of the present invention in non-therapeutic methods.

In one embodiment, the chimeric polypeptide, the nucleic acid, the extracellular vesicle, the population of extracellular vesicles of the present invention is used in vitro or in vivo for assessing the function or biological activity of adiponectin in various biological processes.

In one embodiment, the chimeric polypeptide, the nucleic acid, the extracellular vesicle, the population of extracellular vesicles of the present invention is used with in vitro or in vivo assays to determine the effect of adiponectin in a given biological processes.

### BRIEF DESCRIPTION OF THE DRAWINGS

FIG. 1 shows 5 exemplary constructs of the chimeric polypeptide according to the present invention.

FIG. 2 is a schematic representation of EVs and of the environment of EVs in culture medium (FIG. 2A), of semi-purified EVs (FIG. 2B) and of ultra-purified EVs (FIG. 2C). FIG. 2A: EVs in culture medium are associated with proteins and with contaminants. FIG. 2B: EVs semi-purified by ultracentrifugation are associated with a crown of associated proteins. FIG. 2C: EVs ultra-purified by TFF (tangential flow filtration) and chromatography are associated with proteins anchored in the membrane or tightly associated with the membrane.

FIG. 3 is an immunoblot showing that the chimeric polypeptide of sequence SEQ ID NO: 40 is expressed in cell extracts and in EV. It shows an immunoblot analysis of cells and EV extracts expressing the chimeric polypeptide (3, 5) and of control cell and EV extracts that do not express the chimeric polypeptide (2, 4), wherein the expression of the chimeric polypeptide is detected by antibodies targeting the pilot peptide (2, 3, 4, 5). (1) represents the molecular weight markers.

FIG. 4 is an immunoblot showing that the adiponectin comprised in the chimeric polypeptide within the EV is multimeric. It shows an immunoblot analysis of EV extracts expressing the chimeric polypeptide of sequence SEQ ID NO: 40 (3,4) or of control EV extracts that do not express the chimeric polypeptide (2), wherein the chimeric polypeptide is revealed by antibodies targeting the pilot peptide. The experiments on extracts 2, 3 are performed in reducing conditions (DTT), wherein the experiment on the extract 4 is performed in non-reducing conditions that preserve multimeric structures. (1) represents the molecular weight markers.

FIG. 5 is a combination of three graphs showing the presence of EV markers and of adiponectin at the surface of EV expressing the chimeric polypeptide of sequence SEQ ID NO: 40. The EVs expressing the chimeric polypeptide were subjected to ELISA tests with antibodies targeting CD81 and CD63, two EV markers, and antibodies targeting adiponectin. The anti-adiponectin antibodies do not react with control EV that do not express the chimeric polypeptide.

FIG. 6 is a combination of schemas, immunoblot and table showing the production of adiponectin on EVs semi-purified from culture medium. FIG. 6A is a schematic representation of semi-purified EVs. FIG. 6B is a Western-blot in reducing conditions of semi-purified EVs. Lane 0 corresponds to cells transfected transiently with a DNA construct comprising an empty vector with pilot peptide alone (0). Lane “wt” corresponds to cells transfected transiently with DNA corresponding to wild-type adiponectin. The other lanes correspond to cells transfected transiently with DNA coding for a chimeric adiponectin corresponding to construct 1 (SEQ ID NO: 40, lane 1), construct 2 (SEQ ID NO: 42, lane 2), construct 3 (SEQ ID NO: 41, lane 3), construct 4 (SEQ ID NO: 43, lane 4), construct 5 (SEQ ID NO: 44, lane 5). FIG. 6C is a schematic representation of wild-type adiponectin (wt) and of chimeric adiponectin differently anchored in the membrane and corresponding to chimeric adiponectin with construct 1 to construct 5. FIG. 6D is a table showing the size (in kDa) of proteins identified by a star (*) in FIG. 6B.

FIG. 7 is a combination of a schema and an immunoblot showing the production of multimeric adiponectin on EVs semi-purified from culture medium. FIG. 7A is a schematic representation of semi-purified EVs. FIG. 7B is a Western-blot in non-reducing conditions of semi-purified EVs. Lane 0 corresponds to the analysis of cells transfected transiently with a DNA construct comprising an empty vector with pilot peptide alone (0). Lane “wt” corresponds to the analysis of cells transfected transiently with DNA corresponding to wild-type adiponectin. The other lanes correspond to cells transfected transiently with DNA coding for a chimeric adiponectin corresponding to construct 1 (SEQ ID NO: 40, lane 1), construct 2 (SEQ ID NO: 42, lane 2), construct 3 (SEQ ID NO: 41, lane 3), construct 5 (SEQ ID NO: 44, lane 5).

FIG. 8 is a combination of a schema and immunoblots showing the production of adiponectin on EVs ultra-purified from culture medium. FIG. 8A is a schematic representation of EVs ultra-purified. FIG. 8B displays Western-blots in reducing conditions of the cell extracts and of ultra-purified EVs. Lane (wt) corresponds to wild-type adiponectin. Lane 0 represents control cells, lane 2 adiponectin anchored in the membrane with construct 2, lane (2+wt) the mixture of adiponectin (wt) and of adiponectin anchored in the membrane. FIG. 8C displays the Western-blot in non-reducing conditions of the ultra-purified EVs. Lane (wt) corresponds to wild-type adiponectin. Lane 0 represents control cells, lane 2 adiponectin anchored in the membrane with construct 2, lane (2+wt) the mixture of adiponectin (wt) and of adiponectin anchored in the membrane.

FIG. 9 is a combination of immunoblots, a histogram and graphs showing the characterization of ultra-purified EVs. FIG. 9A shows a Western-blot in reducing/non-reducing conditions of the cell extracts and of ultra-purified EVs with Alix marker as control (left panel: cell extracts in reducing conditions, middle panel: EVs in reducing conditions, right panel: EVs in non-reducing conditions). Lane (wt) corresponds to cells expressing wild-type adiponectin, lane 0 represents adiponectin of control cells and lane 2 represents adiponectin anchored in the membrane with construct 2. FIG. 9B is a combination of two graphs showing the detection by ELISA of CD81 EV marker (left graph) and of adiponectin on the EVs (right graph). FIG. 9C is a histogram showing the quantification of adiponectin by ELISA in EVs from control cells (left column), from cells expressing wild type adiponectin (middle column) and cells expressing the chimeric adiponectin with construct 2 (right column).

FIG. 10 is a combination of two histograms showing the characterization of ultra-purified EVs in terms of concentration (FIG. 10A) and size of the EVs (FIG. 10B). Column (EV “0”) represents EVs of control cells, column (EV “wt”) represents EVs of cells stably expressing wild-type adiponectin and (EV “2”) represents EVs of cell expressing DNA coding for a chimeric adiponectin with Construct 2 (SEQ ID NO: 42).

FIG. 11 is a combination of six graphs showing the ELISA characterization of ultra-purified EVs displaying adiponectin at time zero of production (T0 4° C.) and three months post-production and stored at different conditions (4° C. or −80° C.). It represents ELISA characterization of the conservation of adiponectin (Adpn) and CD81 of ultra-purified EVs, at time zero of production (T0 4° C.) and three months after production under storage at +4° C. and at −80° C. EV “0” represents EVs of control cells. EV “wt” represents EVs of cells stably expressing wild-type adiponectin and EV “2” represents EVs of cell expressing DNA coding for a chimeric adiponectin with Construct 2 (SEQ ID NO: 42).

FIG. 12 is a histogram showing the adiponectin quantification in ultra-purified EVs displaying adiponectin at time zero of production (T0 4° C.) and three months post-production and stored at different conditions (4° C. or −80° C.). It represents the results of a quantitative ELISA analysis, characterizing the conservation of adiponectin (Adpn) of EVs ultra-purified, starting from culture medium of cells stably expressing (EV “wt” and EV “2”) adiponectin, at time zero of production (T0 4° C.) and three months after production under storage at +4° C. and at −80° C. EV “0” represents EVs of control cells. EV “wt” represents EVs of cells stably expressing wild-type adiponectin and EV “2” represents EVs of cell expressing DNA coding for a chimeric adiponectin with Construct 2 (SEQ ID NO: 42).

FIG. 13 is a combination of two histograms showing the size and concentration analysis of ultra-purified EVs displaying adiponectin at time zero of production (T0 4° C.) and three months post-production and stored at different conditions (4° C. or −80° C.). These histograms represent the size (nm) (left panel) and particle concentration (p/ml, particles/milliliter) (right panel) of ultra-purified EVs from culture medium of control cells (EV “0”) and of cells stably expressing (EV “wt” and EV “2”) adiponectin, at time zero of production (T0 4° C.) and three months after production under storage at +4° C. and at −80° C. EV “0” represents EVs of control cells. EV “wt” represents EVs of cells stably expressing wild-type adiponectin and EV “2” represents EVs of cell expressing DNA coding for a chimeric adiponectin with construct 2 (SEQ ID NO: 42).

### EXAMPLES

Adiponectin is one of the most important adipocytokines secreted by adipocyte, and is known to exert beneficial effects in various human conditions, including diabetes, obesity, insulin resistance, cardiovascular disease, inflammatory conditions and cancer. Although adiponectin appears as a promising candidate for drug development for treating various diseases, there are currently no adiponectin therapies available for clinical testing.

Indeed, large-scale production of functional adiponectin is challenging, due to its complexity. Adiponectin is a 244 amino acid cytokine, that includes post-translational modifications and exists in three oligomeric complexes: a low molecular weight (LMW) form, a medium-molecular weight (MMW) and a high molecular weight (HMW) form, which is the most active form of the protein, meaning that functional adiponectin requires the presence of post-translational modifications and proper high multimerization. Bacterial systems lack mammalian protein synthesis machinery and fail to produce functionally active adiponectin, while exploitation of the mammalian culture system for mass production is not a scalable process. In addition, adiponectin has a short half-life in circulation making exogenous administration of recombinant adiponectin a non-feasible approach.

Interestingly, it was found that the highly active HMW adiponectin is present principally in the exosome fraction in serum (Phoonsawat et al., Adiponectin is partially associated with exosomes in mouse serum, BBRC Vol. 448, Issue 3, 2014, Pages 261-266), and that adiponectin mainly distributes at the external surface of extracellular vesicles, as a result of unspecific adsorption of soluble adiponectin (Blandin et al., Extracellular vesicles are stable carriers of adiponectin with insulin-sensitive properties, http://dx.doi.org/10.2139/ssrn.4036824). It was further shown that adiponectin-associated extracellular vesicles mediates insulin sensitizing effects on target cells in vitro and that their injection in high fat diet-fed mice prevent the animals from the development of insulin resistance (Blandin et al., Extracellular vesicles are stable carriers of adiponectin with insulin-sensitive properties, http://dx.doi.org/10.2139/ssrn.4036824).

Although these results are encouraging because they demonstrate that administering adiponectin-associated extracellular vesicles may be a valuable approach to treat diseases associated with adiponectin alteration, such adiponectin-associated extracellular vesicles are not suitable for therapeutic applications. In particular, as demonstrated in the Examples hereinbelow, the ultra-purification of such adiponectin-associated extracellular vesicles induces the removal of the surface adiponectin, leading to ultra-purified extracellular vesicles almost devoid of adiponectin. Thus, there is still a need to provide means enabling a large-scale production of functional adiponectin, i.e. HMW and on extracellular vesicles, suitable for therapeutic applications.

The Inventors have developed herein new chimeric adiponectin that enables to produce, on an industrial scale, extracellular vesicles loaded with functional adiponectin, which are stable several months and which can be useful in therapeutic applications, and in particular, for treating insulin resistance and diabetes, as well as other diseases.

The present invention is further illustrated by the following examples.

### Example 1

### Production of Semi-Purified Extracellular Vesicles Comprising a Chimeric Adiponectin Polypeptide

### Materials and Methods

### Production of Adiponectin and of Extracellular Vesicles Harboring Adiponectin in Mammalian Cells

Extracellular vesicles were produced in HEK293T cells, obtained from American Type Culture Collection (ATCC). Cells were cultured in DMEM supplemented with 5% of heat-inactivated fetal bovine serum (iFBS), 2 mM of GlutaMAX and 5 μg/mL of gentamicin at 37° C. in a 5% CO₂ humidified incubator. HEK293T cells were routinely tested and found negative by MycoAlert™ Mycoplasma detection kit (Lonza Nottingham, Ltd.).

A nucleic acid sequence coding for wild-type adiponectin (SEQ ID NO: 31 or 33) and nucleic acid sequences coding for chimeric adiponectin polypeptides targeted to exosomes (SEQ ID Nos: 40-44) were inserted in an eucaryotic expression vector under the control of a CMV/HTLV chimeric promoter. When necessary, a zeocin encoding resistance gene was added in tandem with the adiponectin-coding nucleic acid sequence and downstream a CMV IRES sequence, allowing for simultaneous expression of zeocin resistance and establishment of stable transfected cell line. These nucleic acid sequences were transfected into HEK293T cells using PEI. When necessary, the selection of a stable transfected cell line was obtained in the presence of 500 μg/mL of zeocin during 15 days.

In order to generate large-scale exosome production, HEK293T transfected cells were plated into cell chambers of 10 trays in 1 L of complete medium. 24 hours later, cultures were fed with medium supplemented with extracellular vesicle-free iFBS and incubated for a further 48 hours.

### Adiponectin-Extracellular Vesicles and Extracellular Vesicle Purification

Cell culture medium was harvested from transfected HEK293T cells and adiponectin-extracellular vesicle isolation was performed as previously described (Taylor & Shah, 2015. Methods. 87:3-10; Desplantes et al., 2017. Sci Rep. 7 (1): 1032; Corso G. et al. 2017. Scientific Reports. 7:11561. DOI: 10.1038/s41598-017-10646-x). Briefly, cell culture supernatant was clarified by two consecutive centrifugations: 10 minutes at 1 300 rpm and 15 minutes at 4 000 rpm, both at 4° C., followed by filtration through 0.22 μm membrane filters. The supernatant was then concentrated by ultra-filtration and diafiltration and load onto either size exclusion chromatography (SEC) or BE-SEC columns (CL2-B or Sephacryl S1000 or Captocore, GE Healthcare). Fractions containing extracellular vesicle biomarkers (CD81 and CD63) were identified by ELISA. Extracellular vesicle fractions containing adiponectin identified by Western-Blot were pooled, concentrated when necessary and used for analysis and injections.

### SDS-PAGE, Western-Blotting and Antibodies

Protein concentration of adiponectin-extracellular vesicles was measured using the BCA assay (Pierce BCA Protein Assay Kit, ThermoFisher Scientific). Adiponectin-extracellular vesicles preparations were lysed and separated by SDS-PAGE on a 4-15% acrylamide gel (4-15% Mini-PROTEAN® TGX Stain-Free™ Gel kit, Bio-Rad) and subsequently transferred onto PVDF membrane. For Western-Blotting in non-reducing conditions, a loading buffer without DTT was used.

Immunodetection of adiponectin was carried out with primary antibodies against either adiponectin (anti-adiponectin monoclonal antibody, clone ABM52A3, Abeomics Ref #10-7597; or anti-adiponectin rabbit polyclonal antibody, Invitrogen Ref. #PA1-054), or anti-Ciloa Pilot Peptide (PP) (in-house antibody raised in rabbit).

Immunodetection of specific extracellular vesicles markers was carried out with primary antibodies against either CD81 (Genetex Ref. #GTX101766), CD63 (Genetex Ref. #GTX132953), Alix (Proteintech #12422-1-AP), syntenin (Fisher Scientific Ref #11326573).

Membranes were then incubated with the corresponding secondary HRP-conjugated antibodies (donkey anti-mouse or anti-rabbit or anti-goat HRP, Jackson ImmunoResearch, Refs. #715-035-150, #711-035-152 or #715-038-147).

The signals were detected using an enhanced chemiluminescence detection kit (Super Signal West Pico Plus; ThermoFischer Scientific; Ref. 34580) and membranes imaged with ChemiDoc Imaging System (Bio-Rad).

These primary antibodies plus the GeneTex (GTX112777) anti-adiponectin polyclonal antibody, as well as respective secondary antibodies, were also used to detect the adiponectin on the surface of adiponectin-extracellular vesicles by ELISA.

### Adiponectin and Extracellular Vesicle Marker Specific IgG ELISA

Extracellular vesicle surface contents in adiponectin, and in CD81- and CD63-specific surface markers were determined by ELISA using some of the above antibodies, and also anti-CD81 (Ancell; Ref. #ANC-302-020) or anti-CD63 (Agro-Bio; Ref. #S12086) antibodies.

Briefly, MaxiSorp ELISA plates (Nunc) were coated with serial 1/2 dilutions (starting from 1 μg) adiponectin-extracellular vesicles in 100 μL in 50 mM sodium carbonate/bicarbonate pH 9.6 buffer per well, overnight at 4° C. Coated plates were washed 3 times with 200 μL of 1×PBS and saturated for 1 hour at 37° C. with 200 μL of 3% BSA in 1×PBS per well. Plates were washed three times with 1×PBS, then incubated in 3% BSA and 5% FBS with primary antibody dilutions (1:500 for adiponectin or 1:10000 for extracellular vesicle-specific markers) for 2 hours at 37° C. This was followed by 3 washes with 200 μL of 1×PBS per well and incubation with 100 μL per well of corresponding secondary HRP conjugated antibody (as specified for Western-Blots above) diluted 1:10000 in 3% BSA in 1×PBS. Following incubation with the secondary antibody, plates were washed 5 times with 200 μL of 1×PBS per well and developed with 100 μL of TMB per well (Bio-Rad; Ref. #R8/R9) for 30 minutes. The reaction was stopped by adding 50 μL of stop solution (2 N sulfuric acid) per well.

The 450 nm-absorbance was read using ClarioStar Plus plate reader (BMG Labtech). The reciprocal endpoint titers were defined as the dilution with the 450 nm OD 3 times higher than the background.

### Results

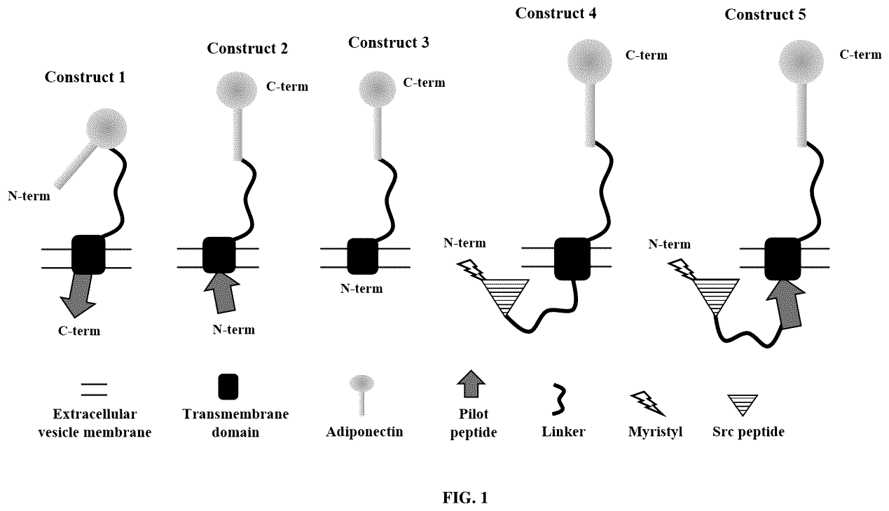

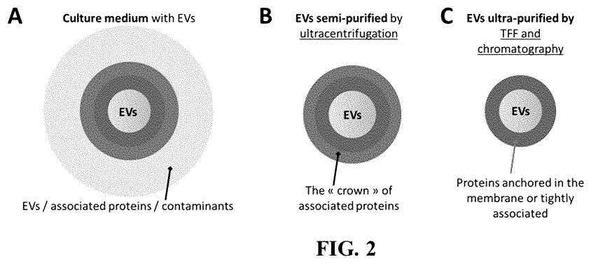

The Inventors have first shown the production of semi-purified EVs comprising chimeric adiponectin with adiponectin in either N-terminal or C-terminal (FIGS. 1 and 2B).

Production of Chimeric Adiponectin with Adiponectin at N-Terminus

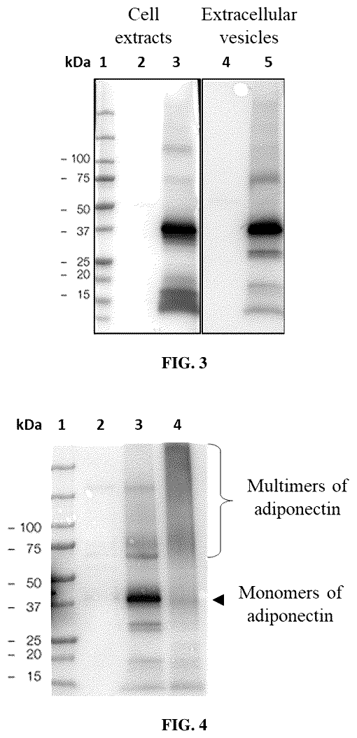

FIG. 3 shows that the chimeric polypeptide with SEQ ID NO: 40, comprising, from N- to C-terminal, adiponectin with its signal peptide (with SEQ ID NO: 33), a (GGGSGGGGS) 3 linker (with SEQ ID NO: 39), a CD8 transmembrane domain (with SEQ ID NO: 37) and a peptide pilot (with SEQ ID NO: 30), is expressed in cells and secreted in extracellular vesicles.

Interestingly, this chimeric polypeptide configuration enabled adiponectin oligomerization in the extracellular vesicles, as detected by immunoblots analysis (FIG. 4).

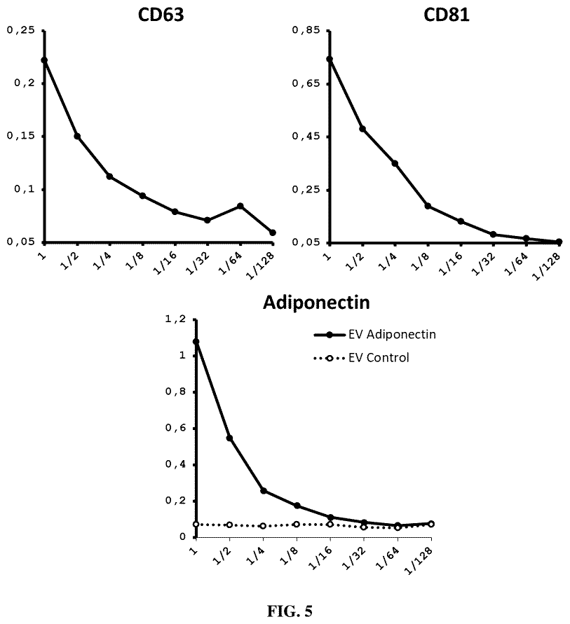

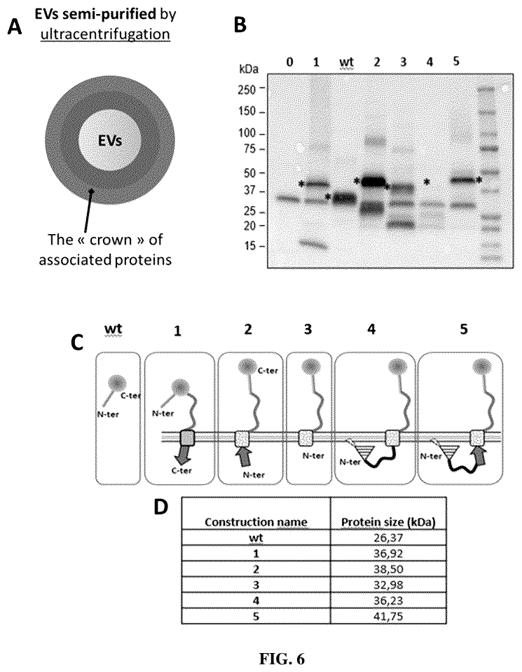

To confirm that adiponectin was expressed at the surface of the extracellular vesicles, we performed ELISA assays to label adiponectin and extracellular vesicle-specific markers in extracellular vesicles expressing the chimeric polypeptide with SEQ ID NO: 40 or in control extracellular vesicles that do not express the chimeric polypeptide. As shown in FIG. 5, the two extracellular vesicle-specific markers (CD63 and CD81), as well as adiponectin, were detected in extracellular vesicles expressing the chimeric polypeptide. The absence of adiponectin detection in control extracellular vesicles confirms the specificity of adiponectin detection in extracellular vesicles expressing the chimeric polypeptide.

Production of Chimeric Adiponectin with Adiponectin in N-Terminus and C-Terminus

The tested constructs are given in FIG. 6C and the size of the proteins revealed by the anti-adiponectin antibody is given in FIG. 6D. The size of adiponectin from cells transfected transiently with DNA corresponding to wild-type adiponectin (wt), chimeric adiponectin with construct 1 (SEQ ID NO: 40), chimeric adiponectin with construct 2 (SEQ ID NO: 42), chimeric adiponectin with construct 3 (SEQ ID NO: 41), chimeric adiponectin with construct 4 (SEQ ID NO: 43), and chimeric adiponectin with construct 5 (SEQ ID NO: 44) is in the range of 26.37 kDa, 36.92 kDa, 38.50 kDa, 32.98 kDa, 36.23 kDa and 41.75 kDa, respectively.

The results further show that the constructs 1, 2, 3 and 5, as well as wt adiponectin express adiponectin in semi-purified EVs. Unexpectedly, the construct 4 do not lead to expression of adiponectin on EVs. To note, the semi-purified EVs harboring the wt adiponectin or the chimeric adiponectin with construct 2 (SEQ ID NO: 42) are the EVs that comprise the largest amount of adiponectin, when analyzed in reducing conditions (FIGS. 6A and 6B).

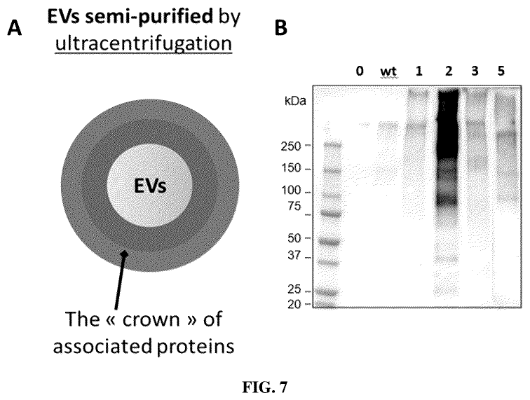

Interestingly, when analyzed in non-reducing conditions (FIG. 7A, FIG. 7B), the results show that the semi-purified EVs harboring the chimeric adiponectin corresponding to construct 2 (SEQ ID NO: 42) comprise a larger amount of highly oligomerized (HMW) adiponectin than EVs harboring wt adiponectin, or EVs harboring the chimeric adiponectin corresponding to construct 3 (SEQ ID NO: 41) or construct 5 (SEQ ID NO: 44).

### Example 2

### Production of Ultra-Purified Extracellular Vesicles Comprising a Chimeric Adiponectin Polypeptide

### Materials and Methods

### Production of Adiponectin and of Extracellular Vesicles Harboring Adiponectin in Mammalian Cells

Extracellular vesicles harboring either wild-type adiponectin, or chimeric adiponectin polypeptide (SEQ ID NO: 42) were produced as described hereinabove.

Production of Adiponectin on EVs Ultra-Purified from Culture Medium

Culture medium of cells stably expressing different DNA constructs: wild-type adiponectin (wt), adiponectin anchored in the membrane with construct 2 (SEQ ID NO: 42) (2), both (2+wt) or control cells (0) was concentrated and purified using TFF and BE-SEC chromatography. The ultra-purified EVs as well as extracts from producer cells were subjected to SDS-PAGE separation in reducing or non-reducing conditions, analyzed by Western-blot and revealed with anti-adiponectin primary antibody followed by a secondary HRP-conjugated antibody, as described hereinabove.

### Characterization of Ultra-Purified EVs

Culture medium of cells stably expressing different DNA constructs: wild-type adiponectin (wt), adiponectin anchored in the membrane (2), or control cells (0) was concentrated and purified using TFF and BE-SEC chromatography. The ultra-purified EVs as well as extracts from producer cells were subjected to SDS-PAGE separation in reducing or non-reducing conditions, analyzed by Western-blot and revealed with anti-Alix (EV marker) and anti-adiponectin primary antibodies followed by a secondary HRP-conjugated antibody.

The presence of an EV marker (CD81) and adiponectin (Adpn) on the surface of EVs was detected by ELISA. The different types of EVs were fixed on ELISA plates in dilutions from 1 to 1/128 (where 1=50 μl of pure EVs) and detected by anti-CD81 antibody or anti-adiponectin antibody followed by a secondary anti-HRP antibody.

The quantity of adiponectin was measured in the EV preparations by quantitative ELISA. EVs were lysed to detect the totality of adiponectin anchored or associated and adiponectin concentration (ng/ml) was measured using commercial ELISA sandwich kit for human adiponectin quantification.

### Results

The Inventors have also shown the production of ultra-purified EVs comprising chimeric adiponectin with adiponectin in C-terminal (FIGS. 1 and 2C).

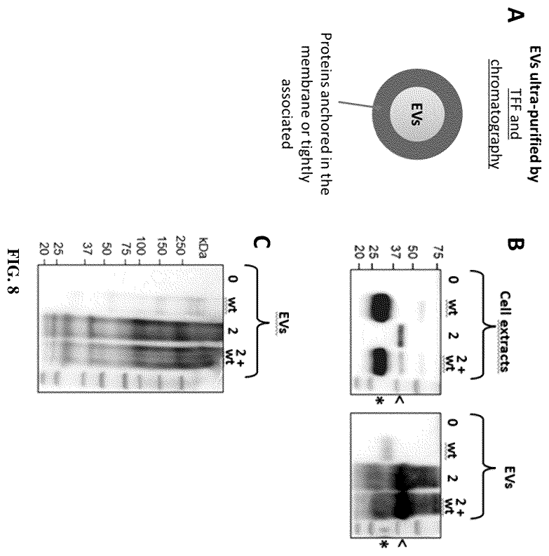

Results of production of ultra-purified EVs and characterization of the ultra-purified EVs are shown in FIG. 8 and FIGS. 9 and 10.

FIG. 8B (left panel) shows that the WT adiponectin (lanes wt and wt+2) is much better expressed in cells than the anchored version (lanes 2 and wt+2). However, when EVs are ultra-purified, the anchored adiponectin (lanes 2 and wt+2) is much more stably targeted (resisting the ultra-purification) onto the EVs than the WT adiponectin (lanes wt and wt+2). This is true when the corresponding constructs are alone (lanes wt and 2) or co-expressed (wt+2) (FIG. 8B, right panel). For EVs ultra-purified and analyzed in non-reducing conditions (FIG. 8C), the construct 2 alone (lane 2) or co-expressed with WT adiponectin (lane 2+wt) is highly oligomerized. Only the construct 2 allows a stable targeting of high quantities of highly oligomerized (HMW) adiponectin onto EVs. In contrast, even though the amount of WT adiponectin is high in cells, it is less present on semi-purified EVs and quite negligible on ultra-purified EVs. These results clearly show that using the chimeric adiponectin construct 2 allows to produce ultra-purified EVs harboring a high amount of highly oligomerized adiponectin at their surface, while ultra-purified EVs obtained from cells transfected with wt adiponectin are almost devoid of adiponectin.

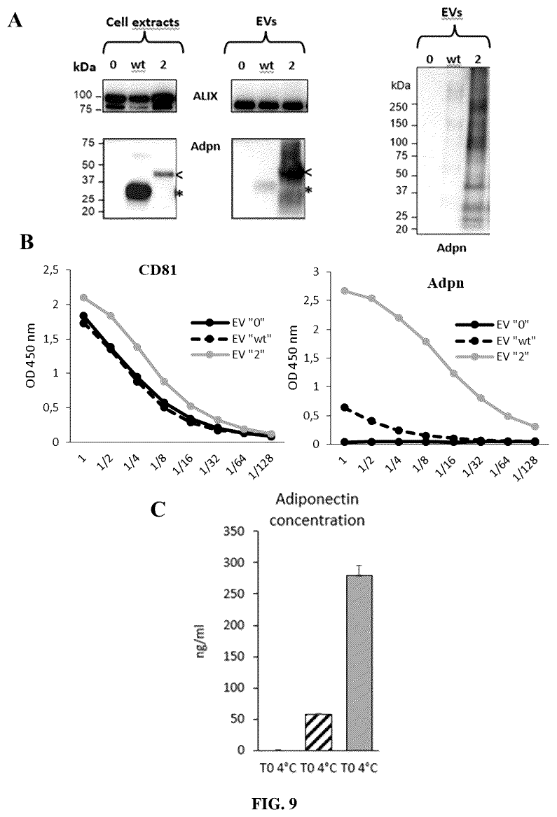

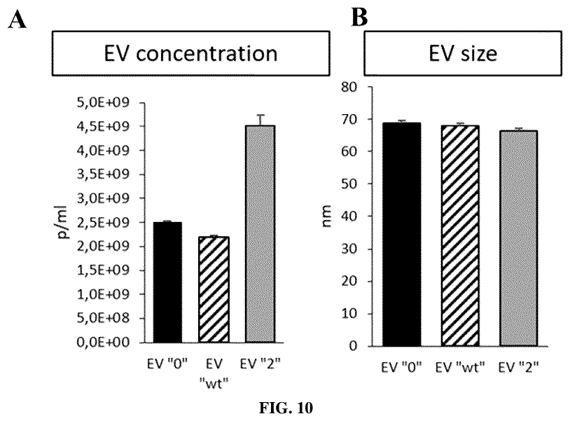

FIGS. 9 and 10 further present the characterization of the ultra-purified EVs. Alix and CD81, two EV markers, and adiponectin are detected in EVs from cells stably expressing wt adiponectin or construct 2 (SEQ ID NO: 42) (FIGS. 9A, 9B). The quantification of adiponectin by ELISA shows that the ultra-purified EVs from cells expressing the construct 2 harbor much more adiponectin than the ultra-purified EVs from cells expressing wt adiponectin (FIG. 9C). These results are further confirmed by analysis with NanoAnalyzer, revealing that ultra-purified EVs from cells expressing the construct 2 that harbor more adiponectin are detected in higher amount than the ultra-purified EVs from cells expressing wt adiponectin (FIG. 10A). As a control, the size of the EVs is similar for both conditions (FIG. 10B).

Altogether, these results show that the chimeric adiponectin with adiponectin at C-terminal, in particular construct 2, enables to obtain ultra-purified EVs harboring high amounts of highly oligomerized (HMW) adiponectin at their surface, which is not the case for the wt adiponectin. As a conclusion, these results clearly demonstrate the advantage of the constructs of the invention, in particular of construct 2, as compared to wt adiponectin.

### Example 3

### Conservation of the Ultra-Purified EVs

### Materials and Methods

ELISA characterization of ultra-purified EVs displaying adiponectin three months post-production and stored at different conditions (4° C. or −80° C.)

The presence of an EV marker (CD81) and adiponectin (Adpn) on the surface of Evs is detected by ELISA shortly after production and purification (T0) or 3 months later upon storage at 4° C. or −80° C. (3 months 4° C. and 3 months −80° C.). The different types of Evs ultra-purified from culture medium of cells stably expressing adiponectin (wt and 2) or control cells (0) are fixed on ELISA plates in dilutions from 1 to 1/128 (where 1=50 μl of pure Evs) and detected by anti-CD81 antibody (top panel) or anti-adiponectin antibody (lower panel) followed by a secondary anti-HRP antibody.

### Adiponectin Quantification in Ultra-Purified EVs Displaying Adiponectin Three Months Post-Production and Stored at Different Conditions (4° C. or −80° C.).

The quantity of adiponectin is measured in the EV preparations by quantitative ELISA shortly after production and purification (TO) or 3 months later upon storage at 4° C. or −80° C. (3 months 4° C. and 3 months −80° C.). The different types of Evs ultra-purified from culture medium of cells stably expressing adiponectin (wt and 2) or control cells (0) are lysed to detect the totality of adiponectin anchored or associated and adiponectin concentration (ng/ml) is measured using commercial ELISA sandwich kit for human adiponectin quantification.

For ELISA quantification, adiponectin concentration was measured using a sandwich ELISA kit (Human Adiponectin/Acrp30 DuoSet ELISA R&D Systems #DY1065-05) following the manufacturer's protocol. Prior to the experiment and to measure the adiponectin quantity in its totality, including the anchored one, the EVs were lysed. Briefly, 1 volume of EVs was incubated with 4 volumes of lysis buffer (NP-40 1%, TNE 1×: Tris 0.1 M, EDTA 1 mM et PMSF 0.25 mM final) during 30 minutes on ice. Following the incubation step Evs were diluted using the Reagent diluent of the kit and 100 μl of lysed EVs were used for the ELISA. Two different dilutions were applied to each EV sample and measurement was performed in technical duplicates for each.

### Size and Concentration Analysis of Ultra-Purified EVs Displaying Adiponectin Three Months Post-Production and Stored at Different Conditions (4° C. or −80° C.)

The size (nm) and concentration (particles per ml: p/ml) of EVs in different preparations is measured using the NanoAnalyzer instrument (NanoFCM).

Ultra-purified EV batches were analysed for their size (nm) and particle concentration in particles per mililiter (p/ml) using the NanoAnalyzer instrument (nanoFCM). The instrument was calibrated with quality control beads (250 nm SiNPs) as well as size standards beads (S16M-Exo) before the analysis, which was performed according to the recommendations. Samples were diluted in 1×PBS for a working sample concentration of around 108 particles/ml and acquired during 1 minute at a rate of maximum 12.000 particles/minute as recommended.

### Results

The Inventors have further demonstrated that the ultra-purified EVs maintain a high amount of adiponectin when stored at 4° C. or −80° C.

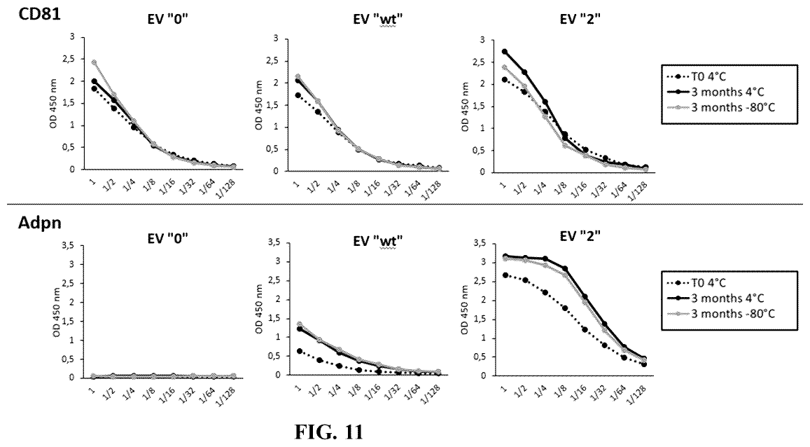

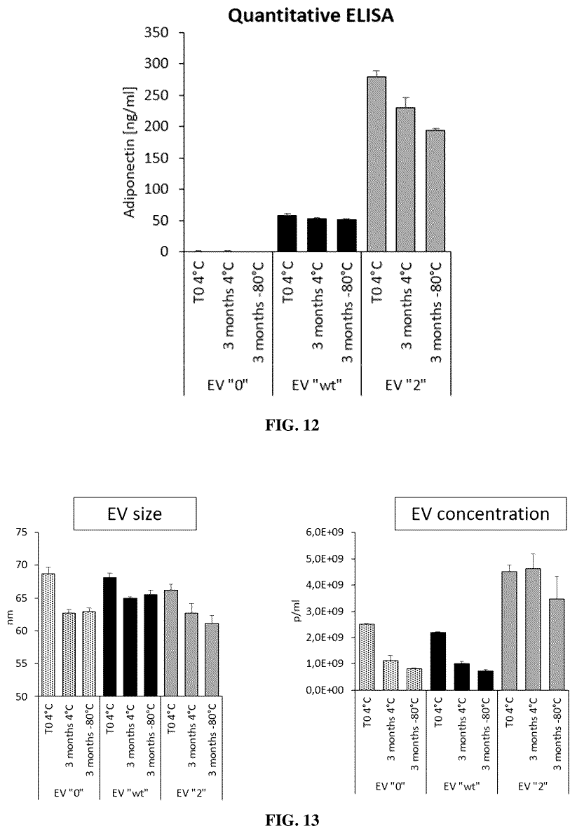

Results are presented in FIGS. 11, 12 and 13. These figures show that the ultra-purified EVs from cells expressing either the construct 2 or wt adiponectin still harbor adiponectin when the EVs are stored during three months at 4° C. or −80° C. (FIG. 11), and that the ultra-purified EVs from cells expressing the construct 2 still contain a higher amount of adiponectin than the EVs from cells expressing wt adiponectin after being stored during three months at 4° C. or −80° C. (FIG. 12). These results are confirmed by analysis with the NanoAnalyzer, which reveals that only negligible variations in EVs concentration are observed when adiponectin is anchored in the EV membrane with construct 2 (FIG. 13, right panel). Regarding the size of the EVs, the variations in size measurements are not significant (FIG. 13, left panel).

1-15. (canceled)

16. A chimeric polypeptide comprising, in any order:
i) an amino acid sequence of adiponectin, and
ii) an amino acid sequence of a transmembrane domain of a transmembrane protein.

17. The chimeric polypeptide according to claim 16, wherein the amino acid sequence of adiponectin comprises the amino acid sequence of wild-type adiponectin.

18. The chimeric polypeptide according to claim 16, further comprising iii) an amino acid sequence of a pilot peptide, wherein said pilot peptide interacts with the Endosomal Sorting Complexes Required for Transport (ESCRT) cellular machinery.

19. The chimeric polypeptide according to claim 16, further comprising at least one linker between the amino acid sequence of adiponectin and the amino acid sequence of the transmembrane domain.

20. The chimeric polypeptide according to claim 18, wherein the chimeric polypeptide comprises, from N- to C-terminal, the components iii), ii) and i).

21. The chimeric polypeptide according to claim 16, further comprising a sub-membrane targeting domain.

22. The chimeric polypeptide according to claim 16, wherein the transmembrane domain is selected from the group comprising the transmembrane domain of CD40L and the transmembrane domain of CD8.

23. The chimeric polypeptide according to claim 22, wherein the transmembrane domain of CD40L comprises an amino acid sequence with SEQ ID NO: 35, and the transmembrane domain of CD8 comprises an amino acid sequence with SEQ ID NO: 37.

24. The chimeric polypeptide according to claim 18, wherein the pilot peptide comprises at least one YxxL motif with SEQ ID NO: 1 or DyxxL motif with SEQ ID NO: 4, and at least one PxxP motif with SEQ ID NO: 8, in which “x” represents any amino acid residue.

25. The chimeric polypeptide according to claim 18, wherein the pilot peptide comprises an amino acid sequence with SEQ ID NO: 30 or a variant thereof,
wherein the variant of SEQ ID NO: 30 retains at least three YxxL and/or DyxxL motifs with SEQ ID NO: 1 and SEQ ID NO: 4, respectively; and at least four PxxP motifs with SEQ ID NO: 8; wherein “x” represents any amino acid residue.

26. A nucleic acid encoding the chimeric polypeptide according to claim 16.

27. An extracellular vesicle comprising the chimeric polypeptide according to claim 16.

28. The extracellular vesicle according to claim 27, wherein the transmembrane domain of the chimeric polypeptide is anchored in the extracellular vesicle lipid bilayer, and wherein the adiponectin of the chimeric polypeptide is exposed at the outer surface of the extracellular vesicle.

29. The extracellular vesicle according to claim 27, wherein the extracellular vesicle is an exosome.

30. A population of extracellular vesicles according to claim 27.

31. The population of extracellular vesicles according to claim 30, further comprising soluble adiponectin.

32. The extracellular vesicle according to claim 27, being purified.

33. A method for treating a disease, disorder or condition in a subject in need thereof, comprising administering the extracellular vesicle according to claim 24, the subject, wherein the disease, disorder or condition is selected from the group comprising diabetes, obesity, insulin resistance, diseases related to insulin resistance or deficiency, hypertension, dyslipidemia, hyperuricemia, atherosclerosis, fibrosis, inflammatory pulmonary diseases, nephrotic disease, sleep apnea, dry eye diseases, inflammatory ocular diseases, gastritis and gastro-esophageal reflux disease, inflammatory bowel disease, pancreatitis, osteoporosis, and inflammatory bone and joint diseases.

34. The method according to claim 33, wherein the extracellular vesicle is partially or totally coated with recombinant adiponectin.

35. The method according to claim 34, wherein the extracellular vesicle is partially or totally coated with recombinant adiponectin fused to lactadherin or a functional C1 and/or C2 domain thereof.


SEQUENCE LISTING (54 sequences)

SEQ ID NO: 1 - skipped

SEQ ID NO: 2 - protein, 4 aa, synthetic construct
  REGION 1..4: note = Pilot peptide
YINL

SEQ ID NO: 3 - protein, 4 aa, synthetic construct
  REGION 1..4: note = Pilot peptide
YSHL

SEQ ID NO: 4 - skipped

SEQ ID NO: 5 - protein, 5 aa, synthetic construct
  REGION 1..5: note = Pilot peptide
DYINL

SEQ ID NO: 6-8 - skipped (3 entries)

SEQ ID NO: 9 - protein, 4 aa, synthetic construct
  REGION 1..4: note = Pilot peptide
PSAP

SEQ ID NO: 10 - protein, 4 aa, synthetic construct
  REGION 1..4: note = Pilot peptide
PTAP

SEQ ID NO: 11 - protein, 40 aa, synthetic construct
  REGION 1..40: note = Pilot peptide
  VARIANT 2: note = Xaa represents any amino acid residue
  VARIANT 3: note = Xaa represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = Xaa represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXSXYXXLXPXXPEXYXXLXPXXPDYXXL

SEQ ID NO: 12 - protein, 37 aa, synthetic construct
  REGION 1..37: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 14: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
PXXPXPXXPXSXYXXLXPXXPEXYXXLXPXXPDYXXL

SEQ ID NO: 13 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
  VARIANT 34: note = X represents any amino acid residue
PXXPXXXXPXXPXSXYXXLXPXXPEXYXXLXPXXPDYXXLXXXX

SEQ ID NO: 14 - protein, 41 aa, synthetic construct
  REGION 1..41: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 14: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
  VARIANT 38: note = X represents any amino acid residue
  VARIANT 39: note = X represents any amino acid residue
  VARIANT 40: note = X represents any amino acid residue
  VARIANT 41: note = X represents any amino acid residue
PXXPXPXXPXSXYXXLXPXXPEXYXXLXPXXPDYXXLXXXX

SEQ ID NO: 15 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXXXXXXXXXXXYXXL

SEQ ID NO: 16 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXXXXXXXXXXDYXXL

SEQ ID NO: 17 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXYXXXXXXXXXYXXL

SEQ ID NO: 18 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXYXXXXXXXXDYXXL

SEQ ID NO: 19 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any amino acid residu
  VARIANT 8: note = X represents any amino acid residue
  VARIANT 9: note = X represents any amino acid residue
  VARIANT 11: note = X represents any amino acid residue
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 20 - protein, 9 aa, synthetic construct
  REGION 1..9: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
PXXPXYXXL

SEQ ID NO: 21 - protein, 10 aa, synthetic construct
  REGION 1..10: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 8: note = X represents any amino acid residue
  VARIANT 9: note = X represents any amino acid residue
PXXPXDYXXL

SEQ ID NO: 22 - protein, 11 aa, synthetic construct
  REGION 1..11: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 9: note = X represents any amino acid residue
  VARIANT 10: note = X represents any amino acid residue
PXXPXYXYXXL

SEQ ID NO: 23 - protein, 12 aa, synthetic construct
  REGION 1..12: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 10: note = X represents any amino acid residue
  VARIANT 11: note = X represents any amino acid residue
PXXPXYXDYXXL

SEQ ID NO: 24 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any one or several amino acid residue(s)
  VARIANT 8: note = X represents any amino acid residue
  VARIANT 9: note = X represents any amino acid residue
  VARIANT 11: note = X represents any one or several amino acid residue(s)
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 25 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 18: note = X represents any amino acid residue
PXXPXXXXPXXPXXXYXXLXPXXPEXYXXLXPXXPDYXXL

SEQ ID NO: 26 - protein, 35 aa, synthetic construct
  REGION 1..35: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
PXXPXPXXPXYXXLXPXXPEXYXXLXPXXPDYXXL

SEQ ID NO: 27 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXXXYXXLXPXXPEXYXXLXPXXPDYXXLXXXX

SEQ ID NO: 28 - protein, 39 aa, synthetic construct
  REGION 1..39: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several mino acid residue(s)
  VARIANT 12: note = X represents any amino acid residue
  VARIANT 13: note = X represents any amino acid residue
  VARIANT 17: note = X represents any amino acid residue
  VARIANT 18: note = X represents any amino acid residue
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
  VARIANT 36: note = X represents any amino acid residue
  VARIANT 37: note = X represents any amino acid residue
  VARIANT 38: note = X represents any amino acid residue
  VARIANT 39: note = X represents any amino acid residue
  VARIANT 15: note = X represents any one or several amino acid residue(s)
  VARIANT 21: note = X represents any one or several amino acid residue(s)
PXXPXPXXPXYXXLXPXXPEXYXXLXPXXPDYXXLXXXX

SEQ ID NO: 29 - DNA, 138 bp, synthetic construct
  misc_feature 1..138: note = CilPP
  source 1..138: mol_type = other DNA
gcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 30 - protein, 45 aa, synthetic construct
  REGION 1..45: note = CilPP encoded by SEQ ID NO: 29
APHFPEISFPPKPDSDYQALLPSAPEIYSHLSPTKPDYINLRPAP

SEQ ID NO: 31 - protein, 226 aa, Homo sapiens
  REGION 1..226: note = Mature form of adiponectin
ETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 32 - protein, 18 aa, synthetic construct
  REGION 1..18: note = Peptide signal
MLLLGAVLLLLALPGHDQ

SEQ ID NO: 33 - protein, 244 aa, Homo sapiens
  REGION 1..244: note = Wild-type adiponectin
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 34 - protein, 25 aa, synthetic construct
  REGION 1..25: note = Transmembrane domain of CD40L
IFMYLLTVFLITQMIGSALFAVYLH

SEQ ID NO: 35 - protein, 52 aa, synthetic construct
  REGION 1..52: note = Transmembrane domain of CD40L
MIETYNQTSPRSAATGLPISMKIFMYLLTVFLITQMIGSALFAVYLHRRLDK

SEQ ID NO: 36 - protein, 26 aa, synthetic construct
  REGION 1..26: note = Transmembrane domain of CD8
IYIWAPLAGICVALLLSLIITLICYH

SEQ ID NO: 37 - protein, 28 aa, synthetic construct
  REGION 1..28: note = Transmembrane domain of CD8
DIYIWAPLAGICVALLLSLIITLICYHR

SEQ ID NO: 38 - protein, 4 aa, synthetic construct
  REGION 1..4: note = Repeat from 1 to 10 times
  REGION 1..4: note = Linker
GGGS

SEQ ID NO: 39 - protein, 9 aa, synthetic construct
  REPEAT 1..9: note = Repeat from 1 to 10 times
  REGION 1..9: note = Linker
GGGSGGGGS

SEQ ID NO: 40 - protein, 353 aa, synthetic construct
  REGION 1..353: note = Construct 1
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTNSGGGSGGGGSGGGSGGGGSGGGSGGGGSGGGGSDIYIWAPLAGICVALLLSLIITLICYHRSRGAPHFPEISFPPKPDSDYQALLPSAPEIYSHLSPTKPDYINLRPAP

SEQ ID NO: 41 - protein, 315 aa, synthetic construct
  REGION 1..315: note = Construct 3
MSRGSMIETYNQTSPRSAATGLPISMKIFMYLLTVFLITQMIGSALFAVYLHRRLDKGGGSGGGGSGGGSGGGGSGGGSGGGGSGGGSGETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 42 - protein, 367 aa, synthetic construct
  REGION 1..367: note = Construct 2
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 43 - protein, 351 aa, synthetic construct
  REGION 1..351: note = Construct 4
MGSSKSKPKDPSQRRRKSRGPGGGSGGGSGGGSGGGSSRGSMIETYNQTSPRSAATGLPISMKIFMYLLTVFLITQMIGSALFAVYLHRRLDKGGGSGGGGSGGGSGGGGSGGGSGGGGSGGGSGETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 44 - protein, 403 aa, synthetic construct
  REGION 1..403: note = Construct 5
MGSSKSKPKDPSQRRRKSRGPGGGSGGGSGGGSGGGSSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 45 - skipped

SEQ ID NO: 46 - protein, 6 aa, synthetic construct
  VARIANT 1: note = M is either present or, when located at the N terminal extremity of the chimeric polypeptide, can be removed in vivo by post-translation processing
  REGION 1..6: note = Sub-membrane targeting domain
  VARIANT 3: note = X represents any amino acid residue
  VARIANT 4: note = X represents any amino acid residue
MGXXKS

SEQ ID NO: 47-48 - skipped (2 entries)

SEQ ID NO: 49 - protein, 11 aa, synthetic construct
  REGION 1..11: note = Sub-membrane targeting domain
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
CKXKXXXXRRR

SEQ ID NO: 50 - protein, 16 aa, synthetic construct
  VARIANT 1: note = M is either present or, when located at the N terminal extremity of the chimeric polypeptide, can be removed in vivo by post-translation processing
  REGION 1..16: note = Sub-membrane targeting domain
MGSSKSKPKDPSQRRR

SEQ ID NO: 51 - protein, 19 aa, synthetic construct
  VARIANT 1: note = M is either present or, when located at the N terminal extremity of the chimeric polypeptide, can be removed in vivo by post-translation processing
  REGION 1..19: note = Src peptide
MGSSKSKPKDPSQRRRKSR

SEQ ID NO: 52 - protein, 23 aa, synthetic construct
  REGION 1..23: note = Sub-membrane targeting domain
  VARIANT 1: note = M is either present or, when located at the N terminal extremity of the chimeric polypeptide, can be removed in vivo by post-translation processing
MGSSKSKPKDPSQRRRKSRGPGG

SEQ ID NO: 53 - protein, 33 aa, synthetic construct
SGGGSGGGGSGGGSGGGGSGGGSGGGGSGGGGS

SEQ ID NO: 54 - protein, 32 aa, synthetic construct
GGGSGGGGSGGGSGGGGSGGGSGGGGSGGGSG